SEQUENCE LISTING (17 sequences)

SEQ ID NO: 1 - protein, 9 aa, synthetic construct
APLQHTAPF

SEQ ID NO: 2 - protein, 9 aa, synthetic construct
APRRRVGGF

SEQ ID NO: 3 - protein, 9 aa, synthetic construct
FPSISTTAW

SEQ ID NO: 4 - protein, 9 aa, synthetic construct
HPQHHAQTF

SEQ ID NO: 5 - protein, 9 aa, synthetic construct
HPQLHAQTF

SEQ ID NO: 6 - protein, 9 aa, synthetic construct
KPDYRCQTY

SEQ ID NO: 7 - protein, 9 aa, synthetic construct
PCCYEKGPE

SEQ ID NO: 8 - protein, 9 aa, synthetic construct
PCCYEKQPE

SEQ ID NO: 9 - protein, 9 aa, synthetic construct
PDDQDTGSE

SEQ ID NO: 10 - protein, 15 aa, synthetic construct
APCSLVSYHGYYILA

SEQ ID NO: 11 - protein, 15 aa, synthetic construct
CYMFATARRKCLTPY

SEQ ID NO: 12 - protein, 15 aa, synthetic construct
HAGYIRIQTSAMFGL

SEQ ID NO: 13 - protein, 15 aa, synthetic construct
IIFVNMRTPYKHHHY

SEQ ID NO: 14 - protein, 15 aa, synthetic construct
KALITQRMLKGLGHY

SEQ ID NO: 15 - protein, 15 aa, synthetic construct
KLFLAKSATRSIVER

SEQ ID NO: 16 - protein, 15 aa, synthetic construct
LARRFSSFRAVTVRC

SEQ ID NO: 17 - protein, 15 aa, synthetic construct
LASCYMFATARRKCL

(19) United States
(12) Patent Application Publication
(10) Pub. No.: US 2026/0186000 A1
(43) Pub. Date: Jul. 2, 2026

(54) ITERATIVE VACCINE DESIGN IN AN ERA OF EMERGING INFECTIOUS DISEASES

(71) Applicant: Board of Regents, The University of Texas System, Austin, TX (US)

(72) Inventors: Nikos Vasilakis, Galveston, TX (US); Peter McCaffrey, Galveston, TX (US); Alice F. Versiani, Galveston, TX (US)

(21) Appl. No.: 19/295,449

(22) Filed: Aug. 8, 2025

Related U.S. Application Data
(60) Provisional application No. 63/681,139, filed on Aug. 8, 2024.

(51) Int. Cl. G01N 33/68; A61K 39/00; A61K 39/12; A61K 49/00; A61P 37/04; G16B 5/00

(52) U.S. Cl. CPC G01N 33/6878; A61K 39/12; A61K 49/0008; A61P 37/04; G16B 5/00; A61K 2039/58; G01N 2333/70539

(57) ABSTRACT

Methods provide a scalable, iterative system for designing multi-epitope vaccines against emerging infectious diseases, particularly alphaviruses. A computational pipeline integrates immune-informatic tools to identify and rank B-cell and T-cell epitopes based on immunogenicity, MHC binding affinity, HLA allele coverage, solubility, and stability. Selected epitopes are validated via peptide microarrays and T-cell immunogenicity assays in human, murine, and nonhuman primate models, ensuring robust immune activation. Final vaccine candidates are optimized for broad viral strain coverage. In vivo mouse studies demonstrate immune response induction and protection against viral challenge. Implemented via a Nextflow-orchestrated, containerized pipeline, executable on high-performance or cloud computing platforms, this system enables rapid, adaptable vaccine design leveraging real-time bio-surveillance data.

### PRIORITY PARAGRAPH

This application claims priority to U.S. Provisional Patent Application Ser. No. 63/681,139 filed Aug. 8, 2024, which is incorporated herein by reference in its entirety

### STATEMENT REGARDING FEDERALLY FUNDED RESEARCH

No federally sponsored research or development was used in the creation of this invention.

### REFERENCE TO SEQUENCE LISTING

A sequence listing is being submitted electronically with this application. The sequence listing is incorporated herein by reference. The sequence listing is contained in the file named “UTMBP0421” which is 15,785 bytes (as measured in Microsoft Windows®) and was created on Aug. 8, 2025.

### FIELD

Aspects are directed generally to medicine and immunology, and more particularly to vaccine development, specifically to methods and compositions for designing multi-epitope vaccines targeting emerging infectious diseases.

### BACKGROUND

Emerging viral diseases have devastated human populations throughout the millennia. Two global pandemics in the form of the Spanish Flu and SARS-COV-2 have each altered the course of human development in enduring albeit in distinct ways. The Spanish Flu killed almost 1% of the global population (Taubenberger and Morens, Emerg Infect Dis 12, 15-22, 2006). SARS-COV-2 congested the global healthcare system and led to a wave of shutdowns that will have long-lasting economic and political consequences that are just now being elucidated (Josephson et al., Nature Human Behaviour 5, 557-65, 2021; Naseer et al., Front Public Health 10, 1009393, 2022). Vaccination plays a crucial role in mitigating the disastrous effects of infectious diseases. The SARS-COV-2 pandemic demonstrated exceptionally rapid vaccine development, achieving a functional vaccine candidate within approximately 300 days from sequencing the viral genome. This rapid development is encouraging. However, it is critical to apply similar focus and resources to emerging pathogens to prevent pandemics.

Infectious disease outbreaks consistently underscore the need to strengthen two key activities: (1) global surveillance of emerging pathogens through coordinated sample collection and sequencing efforts, and (2) the development of a streamlined process for designing candidate vaccines that adapt to real-time bio-surveillance data. While the majority of licensed vaccines are thought to confer protection primarily through the induction of neutralizing antibodies, an increasing body of evidence highlights the underappreciated but critical role of T cells in protective immunity, as they contribute to infection control, reduce disease severity, and promote long-term immune memory—even in the face of waning or variant-evaded antibody responses—as reviewed by Sette and Saphire (Immunity 55, 738-48, 2022). In the case of chikungunya virus (CHIKV), T cells have been implicated in both protective and pathogenic roles, and recent epitope mapping studies have begun to define the landscape of CHIKV-specific T cell targets in humans (Agarwal et al. Nat Commun 16, 5756, 2025). These insights underscore the importance of incorporating T cell-based analyses in vaccine development and immune monitoring pipelines, particularly for diseases where antibody responses alone may not fully capture the complexity of protective immunity. Collectively, these insights highlight the urgent need for enhanced global pathogen surveillance and a streamlined vaccine design process to rapidly address infectious disease outbreaks.

### SUMMARY

One solution to the problems associated with the ability to have a relatively frictionless process to create candidate vaccines that adapt with contemporary bio-surveillance data is described herein with the creation of a system for iterative vaccine design that can be scalable, easy to revise, and broadly accessible for various surveillance initiatives. Specifically, the invention describes a novel computational pipeline for engineering pan-virus (e.g., pan-alphavirus) vaccine candidates targeting genetically diverse viruses. The computational, in vitro, and in vivo processes are demonstrated that allow for a sustainably efficient development pipeline stretching from viral proteome to candidate vaccine payload.

The present invention provides methods, compositions, systems, and kits for designing and producing vaccine candidates targeting emerging infectious diseases, particularly alphaviruses. The invention includes a method for designing vaccine candidates by conducting B-cell epitope profiling, T-cell epitope profiling, and vaccine candidate design. B-cell epitope profiling involves comparing viral proteome sequences against target proteins to identify conserved sequences and performing epitope detection to select B-cell epitopes. T-cell epitope profiling entails predicting MHC-I and MHC-II epitopes, selecting high-affinity epitopes, conducting structural analysis using three-dimensional modeling to assess binding stability, and generating weighted immunogenicity scores based on free solvation energy, surface area, binding affinity, and allele frequency. Vaccine candidates are designed by analyzing scored B-cell and T-cell epitopes to produce compositions comprising multiple epitopes, optimized for broad viral strain coverage and population-specific HLA allele frequencies.

The vaccine candidates target viruses such as alphaviruses (e.g., chikungunya, Mayaro, Venezuelan equine encephalitis, Eastern equine encephalitis, Western equine encephalitis, Ross River, O′nyong-nyong, and Semliki Forest viruses), coronaviruses, orthoflaviviruses, and influenza viruses. The compositions include epitopes derived from viral proteins (e.g., E1, E2, E3, nsp2, nsp3, nsp4) and may incorporate specific peptide sequences (e.g., SEQ ID NO:1-17), formulated with pharmaceutically acceptable carriers, adjuvants, or delivery vehicles such as nanoparticles or viral vectors. The vaccine candidates elicit robust T-cell and B-cell immune responses, characterized by cytokine secretion (e.g., IFN-γ, TNF-α, IL-2) and neutralizing antibody production, providing protection against multiple viral strains.

The method is implemented using a computational pipeline orchestrated by Nextflow with containerized analytical tasks, executable on high-performance computing (HPC) clusters or cloud platforms (e.g., Amazon Web Services, Microsoft Azure, Google Cloud). The pipeline integrates real-time bio-surveillance data to enable iterative vaccine design, allowing rapid updates in response to newly sequenced viral strains. In vitro validation involves peptide microarrays and T-cell immunogenicity assays using sera or peripheral blood mononuclear cells (PBMCs) from human, mouse, or nonhuman primate subjects. In vivo validation includes administering vaccine candidates to animal models to assess immune responses and protection against viral challenges. The invention further provides kits containing the computational pipeline, viral proteome and HLA allele databases, and reagents for validation assays, as well as systems comprising processors, memory, and user interfaces for designing tailored vaccine candidates.

The invention is adaptable to specific species, HLA allele groups, or biochemical properties (e.g., antigenicity, solubility, stability), and is particularly suited for populations at risk due to geographic or occupational exposure. The vaccine compositions are stable, scalable, and effective for preventing or treating infections caused by target viruses, offering a rapid and flexible solution for emerging infectious disease threats.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Definitions—As used herein, the following terms have the meanings ascribed to them unless specified otherwise:

Alphavirus: A genus of positive-sense, single-stranded RNA viruses transmitted primarily by mosquito vectors, including, but not limited to, chikungunya virus (CHIKV), Mayaro virus (MAYV), Venezuelan equine encephalitis virus (VEEV), Eastern equine encephalitis virus (EEEV), Western equine encephalitis virus (WEEV), Ross River virus (RRV), O′nyong-nyong virus (ONNV), Semliki Forest virus (SFV), and other related viruses.

B-cell Epitope: A specific region on an antigen recognized by B-cell receptors or antibodies, capable of eliciting a humoral immune response. B-cell epitopes may be linear (continuous amino acid sequences) or discontinuous (non-contiguous amino acids brought together by protein folding).

T-cell Epitope: A peptide sequence derived from an antigen that binds to major histocompatibility complex (MHC) molecules and is recognized by T-cell receptors, capable of eliciting a cellular immune response. T-cell epitopes include MHC-I epitopes (typically 8-11 amino acids, presented to CD8+ T-cells) and MHC-II epitopes (typically 13-17 amino acids, presented to CD4+ T-cells).

Multi-Epitope Vaccine: A vaccine composition comprising multiple B-cell and/or T-cell epitopes designed to elicit broad immune responses against one or more target pathogens, optimized for immunogenicity, stability, and population coverage.

Immunogenicity: The ability of an antigen, epitope, or vaccine candidate to induce an immune response, including the activation of B-cells, T-cells, and/or the production of antibodies and cytokines such as IFN-γ, TNF-α, and IL-2.

MHC Binding Affinity: The strength of interaction between a peptide epitope and an MHC molecule, typically predicted in silico using tools such as NetMHCpan or NetMHCIIpan, expressed as a binding score or rank (e.g., % Rank_EL).

HLA Allele: A variant of the human leukocyte antigen (HLA) genes encoding MHC molecules, which vary across populations and influence peptide presentation to T-cells. HLA allele frequency refers to the prevalence of specific HLA alleles in a target population, used to optimize vaccine coverage.

Bio-Surveillance Data: Information derived from the monitoring and sequencing of pathogens, including viral proteomes, collected to track the emergence or evolution of infectious diseases.

Computational Pipeline: A series of automated, orchestrated computational processes implemented using software tools (e.g., Nextflow) and containerization (e.g., Docker, Singularity) to perform tasks such as epitope prediction, structural analysis, and vaccine candidate design.

Peptide Microarray: An in vitro assay platform used to evaluate the reactivity of peptide epitopes against sera or immune cells, measuring fluorescence intensity to assess antibody or T-cell responses.

Molecular Dynamics (MD) Simulations: Computational methods used to model the physical movements of atoms and molecules in a peptide-MHC complex, assessing binding stability, free solvation energy, and structural dynamics over time.

Vaccine Candidate: A composition comprising selected epitopes, linkers, and optionally a carrier, adjuvant, or delivery vehicle, designed to elicit protective immune responses against a target pathogen.

Pan-Alphavirus: Referring to a vaccine or epitope designed to provide broad immune protection against multiple alphavirus strains, including both encephalitic and arthritogenic alphaviruses.

In Silico: Referring to computational methods or analyses performed on a computer, including epitope prediction, structural modeling, and immunogenicity scoring.

In Vitro: Referring to experiments conducted outside a living organism, such as peptide microarray assays or T-cell stimulation assays using peripheral blood mononuclear cells (PBMCs).

In Vivo: Referring to experiments conducted within a living organism, such as mouse models used to assess vaccine candidate efficacy and immune responses.

Nanoparticle: A nanoscale delivery vehicle, such as gold nanoparticles or lipid-based nanoparticles, used to enhance the delivery and immunogenicity of vaccine candidates.

Adjuvant: A substance included in a vaccine formulation to enhance the immune response to the vaccine antigens, such as by stimulating innate immunity or promoting antigen presentation.

Flow Cytometry: A technique used to analyze the physical and chemical characteristics of cells, particularly immune cells, to assess T-cell activation (e.g., via surface markers like CD69, CD25, OX-40, CD107a, CD137, CD154) and cytokine secretion (e.g., IFN-γ, TNF-α, IL-2).

Nextflow: A workflow orchestration tool used to manage and execute the computational pipeline, enabling scalable and reproducible vaccine design processes across high-performance computing (HPC) or cloud computing environments.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Schematic of the epitope selection model, illustrating B-cell and T-cell epitope profiling and vaccine candidate design. Green lines indicate data flow, orange lines denote data conversion or splitting, and blue lines represent new data integration. White circles represent processing steps, and gray circles indicate external data sources (multi-FASTA viral proteomes and HLA frequency database.

FIG. 2A-2F. Panoramic evaluation of the reactivity of pan-alphavirus epitopes tested against pre-exposed sera. Peptide microarray was performed with samples originated from human, mice, and nonhuman primates (NHP). Data is presented by median fluorescence after background subtraction. Overlapping pan-alphaviruses epitopes were identified by the relative changes of the fluorescence intensity (z-score) on (A) human Venn diagram and (B) murine Venn diagram. (C) Venn diagram of overlapping pan-alphaviruses epitopes among human and murine responses. (D-E) Heatmaps of B-cluster epitopes fluorescent signal against human and mouse sera, respectively. (F) Representative panel of all 726 tested peptides. Lower panel identifies in silico predictions of overlapping epitopes among all alphavirus's complexes. Unclassified complex: Salmon Pancreas Disease virus (SPDV), Sleeping Disease virus (SDV), Tai Forest virus (TAFV), Madariaga virus (MADV), Caaingua virus (CAAV), Tonate virus (TONV), Southern Elephant Seal virus (SESV), Salmonid Alphavirus (SAV), Mwinilunga virus (MWIV), Eilat virus (EILV). Western Equine Encephalitis Complex: Western equine encephalitis virus (WEEV), Highlands J virus (HJV), Fort Morgan virus (FMV), Buggy Creek virus (BCRV), Whataroa virus (WHAV), Ockelpo virus (OCKV), Sindbis virus (SINV), Kyzylagach virus (KZLV), Babanki virus (BABV), Aura virus (AURV). Venezuelan Equine Encephalitis complex: Venezuelan equine encephalitis virus (VEEV), Bijou Bridge Virus, Trocara virus (TROV), Rio Negro virus (RIOV), Pixuna virus (PIXV), Maramana virus, Mucambo virus (MUCV), Masso das Pedras virus (MDPV), Everglades virus (EVEV), Cabassou virus (CABV). Semliki Forest virus complex: Me Tri virus, Semliki Forest virus (SFV), Sagiyama virus (SAGV), Ross River virus (RV), Igbo-Ora virus, O′nyong-nyong virus (ONNV), Una virus (UNAV), Mayaro virus (MAYV), Getah virus (GETV), chikungunya virus (CHIKV), Bebaru virus (BEBV).

FIG. 3A-3B. Representative superposed trajectory of MD simulations from two different identified peptides bound to MHC-I or MHC-II molecules. (A) Superposed trajectory of MHCI-pep1 bound to the MHC-I (HLA A*02:01:01:01). The presented trajectory comprises 100 superposed frames from a single replica of 200 ns after superposition by the MHC molecule. The peptide is depicted in cartoon and sticks, while the MHC molecule is depicted as a gray cartoon. The left panel shows an upper view of the complex, and the right panel shows a side view (for clarity, part of the MHC molecule is not shown). The MHC P2 and PQ anchoring sites are indicated. (B) same representation as in (A) but showing the trajectory of MHCII-pep1 bound to the MHC-II (HLA-DRA1: DRB1*07:01:01:01). The MHC-II peptide core is highlighted to indicate the peptide regions that fit into the MHC groove. Images of the structures were created using ChimeraX program (Pettersen et al., Protein Science 30, 70-82, 2021).

FIG. 4A-4F. Peptide reactivity on murine PBMCs pre-exposed to alphaviruses. (A) Schematic design of mouse infection with MAYV and TC-83. Blood was collected on days 2 and 14 post-infection for confirmation. Isolated PBMCs were re-stimulated in vitro and evaluated by flow cytometry for T-cell activation (CD4+/CD8+) by surface markers (CD69, CD5, and OX-40) and secretion of cytokines (IFN-γ, TNF-α, and IL-2). (B) PRNT50 confirming the secretion of neutralizing antibodies after infection with both MAYV and TC-83. (C) Relative changes (z-score) of surface markers and cytokines on MAYV pre-exposed PBMCs after re-stimulation with peptides. (D) Heatmap with the representation of p-values obtained by 2-way ANOVA statistical analysis of C. (E) Relative changes (z-score) of surface markers and cytokines on TC-83 pre-exposed PBMCs after re-stimulation with peptides. (F) Heatmap with the representation of p-values obtained by 2-way ANOVA with Dunnett's multiple comparisons test of E.

FIG. 5A-5O. Immunogenic profiles of HLA-typed PBMCs from healthy donors after in vitro expansion with pan-alphaviruses peptides. (A) Schematic design of peptide-specific in vitro T-cell differentiation and clonal expansion protocol described in (Cimen Bozkus et al., STAR Protocols 2, 100758, 2021). (B) Donors descriptives by ethnic group, separated in Caucasian, Hispanic, African, and Asian-respectively. The following plots represents the z-score (C-F) and p-value (G-K) of stimulated T-cells by ethnic group. (L-O) Clustering tree of stimulated T-cells. External colors identify different clusters, internal colors identify stimulated T-cell and activation biomarkers on each cluster, and circle size is related to yield of stimulated cells.

FIG. 6A-6C. Immunoprofile of activated T-cells on human PBMCs followed by in vitro stimulation with alphaviruses peptides. PBMCs were collected from donors previously exposed to alphaviruses vaccines TC-83, TSI-GSD 104/inactivated PE-6 strain, and/or TSI-GSD 210/inactivated CM-4884 strain. (A) Experimental design of PBMC stimulation. Isolated PBMCs were re-stimulated in vitro and evaluated by flow cytometry for T-cell activation (CD4+/CD8+) by surface markers (CD107a, CD137, and CD154) and secretion of cytokines (IFN-γ, TNF-α, and IL-2). (B) Relative changes (z-score) of surface markers and cytokines on pre-exposed PBMCs after re-stimulation with peptides. (C) Heatmap with the representation of p-values obtained by 2-way ANOVA statistical analysis of B.

FIG. 7A-7C. Heatmap analysis of the peptide microarray intensity signal tested against different sera pools. (A) Human sera pools. (B) Mouse sera pools. (C) Nonhuman primate sera pools. Y-axis indicates peptides, X-axis indicates sera pool tested.

FIG. 8A-8B. MD simulation analysis of MHC-I bound peptides. (A) Peptide backbone RMSD (in Å) calculated after trajectory superposition by the MHC in five independent replicas. The simulation duration for each replica is 200 nanoseconds, and each line color corresponds to a different replica. (B) Carbon beta (CB) deviation for the peptide positions P2 (upper row) or PQ (bottom row) in comparison to the same positions in a peptide complex (PDB ID: 5hhn) during the simulations for each peptide.

FIG. 9. MD simulation analysis of MHC-I bound reference peptides. Peptide backbone RMSD (in Å) calculated after trajectory superposition by the MHC in five independent replicates. The time of simulation is 200 nanoseconds, and each line color corresponds to a different replicate. The reference PDB structures used for the simulations are: 7u21, 1duz and 5e00. The median RMSD of the last half of the simulations considering all replicas is 1.2, 1.9 and 1.2 Å, respectively.

FIG. 10. MD simulation analysis of MHC-II bound peptides. (A) Peptide backbone RMSD (in Å) calculated after trajectory superposition by the MHC in five independent replicas. The time of simulation is 200 nanoseconds, and each line color corresponds to a different replicate. Of note the peptide backbone deviation also includes peptide N and C-terminal regions outside the binding score.

FIG. 11A-11L. T-cell activation surface markers and cytokine secretion after pre-exposed murine PBMCs are stimulated with pan-alpha peptides. PBMCs from infected mice were stimulated with the vehicle control (UT, untreated), positive control (PMA), and test alphaviruses peptides. (A) MHC-I peptides, IFN-gamma+ cells. (B) MHC-II peptides, IFN-gamma+ cells. (C) MHC-I peptides, TNF-alpha+ cells. (D) MHC-II peptides, TNF-alpha+ cells. (E) MHC-I peptides, IL-2+ cells. (F) MHC-II peptides, IL-2+ cells. (G) MHC-I peptides, CD69+ cells. (H) MHC-II peptides, CD69+ cells. (I) MHC-I peptides, CD25+ cells. (J) MHC-II peptides, CD25+ cells. (K) MHC-I peptides, OX-40+ cells. (L) MHC-II peptides, OX-40+ cells. Percentage of T-cells was measured by flow cytometry. Data were represented as mean±standard deviation (SD). p-values obtained by 2-way ANOVA with Dunnett's multiple comparisons test. (*) p<0.5; (**) p<0.01; (***) p>0.001; (****) p<0.0001.

FIG. 12A-12F. Cytokine induction on human PBMCs followed by in vitro T-cell expansion with alphaviruses peptides. PBMCs from a healthy donor were expanded following stimulation with the vehicle control (UT, untreated), positive control (PMA), CEFT (which contains known viral epitopes), and test alphaviruses peptides. Donors were grouped by ethnicity: Caucasian, Hispanic, African, and Asian. (A) MHC-I peptides, IFN-gamma+ cells. (B) MHC-I peptides, TNF-alpha+ cells. (C) MHC-I peptides, IL-2+ cells. (D) MHC-II peptides, IFN-gamma+ cells. (E) MHC-II peptides, TNF-alpha+ cells. (F) MHC-II peptides, IL-2+ cells. Left graphs indicate CD4+ cells, right graphs indicate CD8+ cells. Percentage of T-cells was measured by flow cytometry. Data were represented as mean±standard deviation (SD). p-values obtained by 2-way ANOVA with Dunnett's multiple comparisons test. (*) p<0.5; (**) p<0.01; (***) p>0.001; (****) p<0.0001.

FIG. 13A-13F. Surface markers induction on human PBMCs followed by in vitro T-cell expansion with alphaviruses peptides. PBMCs from a healthy donor were expanded following stimulation with the vehicle control (UT, untreated), positive control (PMA), CEFT (which contains known viral epitopes), and test alphaviruses peptides. Donors were grouped by ethnicity: Caucasian, Hispanic, African, and Asian. (A) MHC-I peptides, CD107a+ cells. (B) MHC-I peptides, CD137+ cells. (C) MHC-I peptides, CD154+ cells. (D) MHC-II peptides, CD107a+ cells. (E) MHC-II peptides, CD137+ cells. (F) MHC-II peptides, CD154+ cells. Left graphs indicate CD4+ cells, right graphs indicate CD8+ cells. Percentage of T-cells was measured by flow cytometry. Data were represented as mean #standard deviation (SD). p-values obtained by 2-way ANOVA with Dunnett's multiple comparisons test. (*) p<0.5; (**) p<0.01; (***) p>0.001; (****) p<0.0001.

FIG. 14A-14C. Immunogenic profiles of PBMCs from healthy donors after in vitro expansion with pan-alphaviruses peptides. (A) Schematic design of peptide-specific in vitro T-cell differentiation and clonal expansion protocol described in Cimen Bozkus et al., STAR Protocols 2, 100758, 2021. The following plots represent a Principal Components Analysis (PCA) performed on each of the 17 epitopes. In these plots, the first two principal components (PC1 and PC2) constitute the y and x axes respectively and each point represents a single epitope. Input to PCA analysis were cytokine and T-cell activation markers measurements using donor PBMCs. In total there were six signatures measured: CD107a, CD137, CD154, IFN-gamma, TNF, and IL-2 for alongside CD4 and CD8, compressing 6 dimensions into two principal components. The different plots show these data filtered by (B) CD4 and CD8, as well as (C) ethnicity or virus complex prior to PCA.

FIG. 15A-15F. Cytokine induction on human PBMCs followed by in vitro stimulation with alphaviruses peptides. PBMCs from donors previously exposed to alphaviruses vaccines were stimulated with the vehicle control (UT, untreated), positive control (PMA), CEFT (which contains known viral epitopes), and test alphaviruses peptides. (A) MHC-I peptides, IFN-gamma+ cells. (B) MHC-I peptides, TNF-alpha+ cells. (C) MHC-I peptides, IL-2+ cells. (D) MHC-II peptides, IFN-gamma+ cells. (E) MHC-II peptides, TNF-alpha+ cells. (F) MHC-II peptides, IL-2+ cells. Left graphs indicate CD4+ cells, right graphs indicate CD8+ cells. Percentage of T-cells was measured by flow cytometry. Data were represented as mean±standard deviation (SD). p-values obtained by 2-way ANOVA with Dunnett's multiple comparisons test. (*) p<0.5; (**) p<0.01; (***) p>0.001; (****) p<0.0001.

FIG. 16A-16F. Surface markers induction on human PBMCs followed by in vitro stimulation with alphavirus peptides. PBMCs from donors previously exposed to alphaviruses vaccines were stimulated with the vehicle control (UT, untreated), positive control (PMA), CEFT (which contains known viral epitopes), and test alphavirus peptides. (A) MHC-I peptides, CD107a+ cells. (B) MHC-I peptides, CD137+ cells. (C) MHC-I peptides, CD154+ cells. (D) MHC-II peptides, CD107a+ cells. (E) MHC-II peptides, CD137+ cells. (F) MHC-II peptides, CD154+ cells. Left graphs indicate CD4+ cells, right graphs indicate CD8+ cells. Percentage of T-cells was measured by flow cytometry. Data were represented as mean±standard deviation (SD). p-values obtained by 2-way ANOVA with Dunnett's multiple comparisons test. (*) p<0.5; (**) p<0.01; (***) p>0.001; (****) p<0.0001.

FIG. 17. Selection of species-specific candidates based on in silico and in vitro combined score. Pipeline workflow for final vaccine candidate design and optimization.

FIG. 18. Schematic showing Experimental Overview of Initial In Vivo Test of PanAlpha Vaccine. Male and female CD1 mice were IM vaccinated with PanAlpha nanoparticles, gold particles, 0.9% sodium chloride (diluent) or TC-83 (experimental VEEV vaccine) on days 0 and 21. Mice were monitored for health and weights. Blood was taken for antibody and viremia quantification. Post challenge, weight, health and survival were recorded.

FIG. 19. Weight changes and survival post VEEV challenge. (A) Weight changes post vaccination. Arrow at day 21 post vaccination shows boost. (B) Weight change post VEEV challenge. (C) Survival post challenge. Astrix shows significant difference between PanAlpha+ Adjuvant and diluent, PanAlpha-Adjuvant or TC-83 (Logrank test).

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

Recent computational biology and machine learning advancements have revolutionized our ability to generate rationally designed vaccines. Epitope-based designs allow easy synthesis, plasticity on the delivery methods and formulations, well-defined overall stability and solubility, and are biologically safe. In a now-called immune-informatic approach, in silico tools are leveraged to select short immunogenic peptide fragments with the ability to elicit strong and targeted immune responses and allow the amplification of the candidate antigen coverage. Similar approaches were used for several infectious agents prototype candidates, such as Leishmania (Rabienia et al., PLOS One 19, e0295495, 2024; Silva et al., Frontiers in Immunology 7, 2016), hepatitis C (He et al., Scientific Reports 5, 12501, 2015), SARS-COV-2 (Yashvardhini et al., Can J Infect Dis Med Microbiol, 6627141, 2021), Mayaro virus (Khan et al., Infection, Genetics and Evolution 73, 390-400, 2019), Marburg Virus (Sami et al., ACS Omega 6, 32043-32071, 2021), and influenza (Staneková et al., Virology Journal 7, 351, 2010). One of the main features of this approach resides on the multi-target designs to enhance immunogenicity, reduce toxicity or allergenicity, and the ability to direct the response towards specific B or T-cell stimulation. The efficiency of the candidate vaccine to trigger an effective immune response can also be assessed by an in silico immune stimulation using structural modeling, refinement, and validation tools (Motamedi et al., PLOS ONE 18, e0275237, 2023; Yang et al., Scientific Reports 11, 3238, 2021).

Regarding potential emerging pathogens, alphaviruses are positive-sense, single-stranded RNA viruses transmitted by mosquito vectors. Alphaviruses causing significant human disease are generally classified into one of two groups dependent upon the type of disease they cause—encephalitic or arthritogenic. Arthritogenic alphaviruses like chikungunya (CHIKV) and Mayaro (MAYV) viruses cause inflammatory musculoskeletal and joint-associated diseases, often with chronic (months to years) pain impacting quality of life. Encephalitic alphaviruses include Eastern (EEEV), Western (WEEV), and Venezuelan (VEEV) equine encephalitis viruses that, in some patients, result in neurological disease (Griffin, in Fields Virology, D. M. Knipe, P. Howley, Eds. (Lippincott Williams & Wilkins, 2013), pp. 652-686; Chen et al., J Gen Virol 99, 761-62, 2018). The worldwide geographical distribution of viruses, the diversity of mosquito vectors, the wide range of vertebrate reservoir hosts (ranging from rodents to birds), and socioeconomic and ecological factors, provide opportunities for infection or emergence into susceptible animal and human populations (reviewed in Baker et al., Nat Rev Microbiol 20, 193-205, 2022; Weaver et al., Antiviral Res 85, 328-45, 2010). Although major efforts were applied to develop an effective vaccine against some of the main alphaviruses in the past fifty years, approaches have led to some degree of success regarding a full-protective response against different alphaviruses (reviewed in Kim et al., Nature Reviews Microbiology 21, 396-407, 2023), leading to the first FDA-approved CHIKV vaccine on December 2023 (Mullard et al., Nat Rev Drug Discov 23:8, 2024). However, the co-circulation of viruses in different areas of the globe, the outbreak reports in recent years, the high mortality and/or morbidity of some of the family representants, and the lack of antiviral drugs or specific therapeutics (Azar et al., Microorganisms 8, 1167, 2020), put these viruses on the central spotlight for the development of multi-epitope immunogen candidates.

The COVID-19 pandemic demonstrated the importance of a rapid and adaptable vaccine development infrastructure. What might have previously taken years was accomplished within months, allowing for early vaccination programs to be implemented. While SARS-COV2 captured global attention, it is important to recognize that there is an expansive and diverse ecosystem of viruses worldwide, many of which could achieve human transmission and lead to another pandemic. Over the last century, public health authorities have raised concerns about the emergence or reemergence of arthropod-borne zoonotic agents-particularly viruses from the genus Alphavirus, which significantly impact animal and human health.

The lengthy literature on alphavirus vaccines goes back to the 70s. Several approaches were tested, with different degrees of success. Live-attenuated candidates were developed by introducing mutations or deletions in structural and nonstructural proteins (Gorchakov et al., J Virol 86, 6084-96, 2012; Edelman et al., Am J Trop Med Hyg 62, 681-85, 2000; Paessler et al., J Virol 77, 9278-86, 2003; Rossi et al., PLOS Negl Trop Dis 9, e0003797, 2015; Trobaugh et al., PLOS Pathog 15, e1007584, 2019; Tucker et al., J Virol 65, 1551-57, 1991; Plante et al., PLOS Pathog 7, e1002142, 2011; Roques et al., JCI Insight 2, 2017). On the other hand, infectious virions were chemically treated to generate inactivated candidates (Pittman et al., Vaccine 14, 337-43, 1996; Robinson et al., Mil Med 141, 163-66, 1976; Tiwari et al., Vaccine 27, 2513-22, 2009; Foster et al., Comp Immunol Microbiol Infect Dis 6, 31-37, 1983). Formalin-inactivated vaccines require multiple boosts to achieve and maintain detectable neutralizing antibody titers in human recipients and provide incomplete protection against aerosol challenges in animal models (Wolfe et al., The American Journal of Tropical Medicine and Hygiene 91, 442-50, 2014). Attenuated mutants derived from these inactivated vaccines showed improved mucosal protection (Hart et al., Vaccine 18, 3067-75, 2000), but studies described the potential for virulence reversion given that the attenuation relies on only two-point mutations (Davis et al., Virology 212, 102-10, 1995). Some candidates, such as the TC-83 VEEV vaccine, have received the Investigational New Drug (IND) status, but its high reactogenicity restricted the usage to at-risk personnel (Pittman et al., Vaccine 14, 337-43, 1996). Other essential information relates to the subsequential vaccination against alphaviruses and the cross-reactivity among immune responses. Multiple studies have now shown that there is potential for immune interference when different vaccines are subsequently applied to protect against VEEV, EEEV, and WEEV (Wolfe et al., The American Journal of Tropical Medicine and Hygiene 91, 442-50, 2014; Pittman et al., Vaccine 27, 4879-82, 2009; Reisler et al., Vaccine 30, 7271-77, 2012). Pittman and collaborators indicate that vaccinating humans against EEEV and WEEV with inactivated vaccines may interfere with subsequent neutralizing antibody response to the live attenuated VEEV (Pittman et al., Vaccine 27, 4879-82, 2009). This evidence indicates the necessity of developing a multivalent and protective candidate that surpasses individual vaccines' immune response.

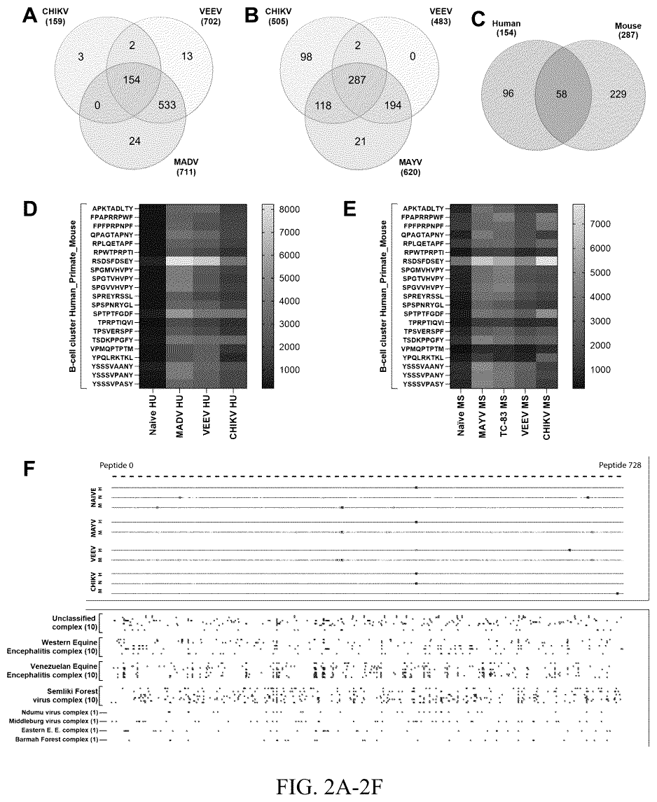

Most of the candidates described in literature are based on the structural protein C-E3-E2-6K-E1 complex. The recently FDA-approved CHIKV candidates, IXCHIQ and VIMKUNYA, are a live-attenuated with a deletion on the nsP3 gene and can generate high levels of neutralizing antibodies (Schneider et al., The Lancet 401, 2138-47, 2023) and virus-like particle vaccines (Hamer et al., Lancet Microbe 6, 101000, 2025), respectively. Other candidates in clinical phases also contain genes that correspond to structural proteins. Our pipeline ideally identified the most suitable epitopes among proteins E1 and E2 for a broad pan-alpha response. However, the list of most reactive overlapping epitopes also included peptides from E3, nsp2, nsp3, and nsp4 (FIG. 2, Table 1). Literature corroborates our findings with non-infectious viral-like particle (VLP) vaccines and viral-vectored candidates that have shown high immunogenicity against structural proteins (Roques et al., JCI Insight 2, 2017; Akahata et al., Nat Med 16, 334-38, 2010; Goo et al., J Infect Dis 214, 1487-91, 2016; Ko et al., Science Translational Medicine 11, eaav3113, 2019; Brandler et al., Vaccine 31, 3718-25, 2013; Ramsauer et al., Lancet Infect Dis 15, 519-27, 2015; Wang et al., Vaccine 29, 2803-09, 2011; Henning et al., Front Immunol 11, 598847, 2020) but highlights the necessity of suitable vaccine vectors for a boost on the immune cellular response (Tatsis et al., Blood 110, 1916-1923 (2007).

TABLE 1
Final selected MHC-I and MHC-II epitopes.

| | | T-cell | B-cell | Classification/ | | Protein |
|---|---|---|---|---|---|---|
| ID | Sequence | type | cluster? | Species | Root | position |
| MHC-I Pep1 | APLQHTAPF | MHC-I | No | top_ranked | Chikungunya virus; | E1 |
| | (SEQ ID NO: 1) | | | human_primate_ | O'nyong-nyong virus; Igbo | |
| | | | | mouse | Ora virus | |
| MHC-I Pep2 | APRRRVGGF | MHC-I | No | top_ranked | Madariaga virus; Eastern | nsp3 |
| | (SEQ ID NO: 2) | | | human_primate_ | equine encephalitis virus | |
| | | | | mouse | | |
| MHC-I Pep3 | FPSISTTAW | MHC-I | No | top_ranked | Barmah Forest virus | E1 |
| | (SEQ ID NO: 3) | | | human_primate_ | | |
| | | | | mouse | | |
| MHC-I Pep4 | HPQHHAQTF | MHC-I | No | top_ranked | Venezuelan equine | E1 |
| | (SEQ ID NO: 4) | | | human_primate_ | encephalitis virus; Tonate | |
| | | | | mouse | virus; Mucambo virus; | |
| | | | | | Cabassou virus; Everglades | |
| | | | | | virus | |
| MHC-I Pep5 | HPQLHAQTF | MHC-I | No | top_ranked | Venezuelan equine | E1 |
| | (SEQ ID NO: 5) | | | human_primate_ | encephalitis virus; Tonate | |
| | | | | mouse | virus; Mucambo virus; | |
| | | | | | Cabassou virus; Everglades | |
| | | | | | virus | |
| MHC-I Pep6 | KPDYRCQTY | MHC-I | No | top_ranked | Una virus; Semliki Forest | E1 |
| | (SEQ ID NO: 6) | | | human_primate_ | virus; Trocara virus; | |
| | | | | mouse | Sagiyama virus; Getah | |
| | | | | | virus; Yada yada virus; | |
| | | | | | Caaingua virus | |
| MHC-I Pep7 | PCCYEKGPE | MHC-I | Yes | no_mouse_no_ | Chikungunya virus; | E3 |
| | (SEQ ID NO: 7) | | | primate_no_human | Mayaro virus; Ross River | |
| | | | | | virus; O'nyong-nyong | |
| | | | | | virus; Igbo Ora virus | |
| MHC-I Pep8 | PCCYEKQPE | MHC-I | Yes | no_mouse_no_ | Chikungunya virus; Ross | E3 |
| | (SEQ ID NO: 8) | | | primate_no_human | River virus; O'nyong- | |
| | | | | | nyong virus; Sagiyama | |
| | | | | | virus; Getah virus | |
| MHC-I Pep9 | PDDQDTGSE | MHC-I | Yes | no_mouse_no_ | Sleeping disease virus; | nsp4 |
| | (SEQ ID NO: 9) | | | primate_no_human | Salmonid alphavirus | |
| | | | | | subtype 3 | |
| MHC-II Pep1 | APCSLVSYHGYYILA | MHC-II | Yes | human_top_mouse_ | encephalitis virus; Pixuna | E2 |
| | (SEQ ID NO: 10) | | | bottom | Rio Negro virus; | |
| | | | | | Venezuelan equine | |
| | | | | | virus; Madariaga virus; | |
| | | | | | Eastern equine encephalitis | |
| | | | | | virus | |
| MHC-II Pep2 | CYMFATARRKCLTPY | MHC-II | No | top_ranked_human_ | Bebaru virus; Una virus; | E2 |
| | (SEQ ID NO: 11) | | | mouse | Chikungunya virus; | |
| | | | | | Middelburg virus; Mayaro | |
| | | | | | virus; Semliki Forest virus; | |
| | | | | | Western equine | |
| | | | | | encephalitis virus | |
| MHC-II Pep3 | HAGYIRIQTSAMFGL | MHC-II | No | top_ranked_human_ | Venezuelan equine | E2 |
| | (SEQ ID NO: 12) | | | mouse | encephalitis virus; Tonate | |
| | | | | | virus; Mucambo virus; | |
| | | | | | Cabassou virus; Madariaga | |
| | | | | | virus; Eastern equine | |
| | | | | | encephalitis virus | |
| MHC-II Pep4 | IIFVNMRTPYKHHHY | MHC-II | No | top_ranked_human_ | Venezuelan equine | nsp2 |
| | (SEQ ID NO: 13) | | | mouse | encephalitis virus; | |
| | | | | | Madariaga virus; Eastern | |
| | | | | | equine encephalitis virus | |
| MHC-II Pep5 | KALITQRMLKGLGHY | MHC-II | No | top_ranked_human_ | Rio Negro virus; | nsp4 |
| | (SEQ ID NO: 14) | | | mouse | Venezuelan equine | |
| | | | | | encephalitis virus; Pixuna | |
| | | | | | virus; Madariaga virus; | |
| | | | | | Eastern equine encephalitis | |
| | | | | | virus | |
| MHC-II Pep6 | KLFLAKSATRSIVER | MHC-II | No | top_ranked_human_ | Bebaru virus | nsp2 |
| | (SEQ ID NO: 15) | | | mouse | | |
| MHC-II Pep7 | LARRFSSFRAVTVRC | MHC-II | No | top_ranked_human_ | Mayaro virus; Ross River | E2 |
| | (SEQ ID NO: 16) | | | mouse | virus; Sagiyama virus; | |
| | | | | | Getah virus | |
| MHC-II Pep8 | LASCYMFATARRKCL | MHC-II | No | top_ranked_human_ | Una virus; Middelburg | E2 |
| | (SEQ ID NO: 17) | | | mouse | virus; Mayaro virus; | |
| | | | | | Semliki Forest virus; Ross | |
| | | | | | River virus; Sagiyama | |
| | | | | | virus; Getah virus | |

Although several candidates currently in clinical trials are live-attenuated candidates, it is essential to stress the possibility of risk of reversion to a pathogenic virus, which can limit the broad application of this immunogen. With this, subunit vaccines present exceptional safety of the vaccinees and ease of production, where the immunoinformatic in silico approach can determine potential bottlenecks and optimize a new candidate (Rawat et al., Vaccines (Basel) 11, 2023). This is especially important in the context of the current global population, which includes a vast and variable genetic pool, and high numbers of immunosuppressed individuals, infants, and the elderly (Pollard et al., Nature Reviews Immunology 21, 83-100, 2021). Thus, strategies that increase the capacity and coverage of subunit vaccines are enormously important. Some authors aim to compensate for the population's genetic variation and improve vaccine coverage even for pathogens with well-established immunogens-which is the case of tuberculosis (Sharma et al., Scientific Reports 11, 13836, 2021). Therefore, our pipeline included the genetic background of the population with the most risk of infection for different alphaviruses as a constraint for the epitope selection. Our results show that Hispanic and African donors presented more robust activation of T-cells compared to Caucasian donors-which can be explained by the most frequent HLA-haplotypes observed in South America-population used as target in the pipeline. Additionally, Khor and collaborators have shown an association between HLA and antibody response after vaccination against SARS-COV-2 (Khor et al., Vaccines (Basel) 10, 2022). In another SARS-COV-2 vaccine evaluation, Bertinetto et al. discuss that HLA significantly influences cellular and humoral responses (Bertinetto et al., HLA 102, 301-15, 2023). The difference between the epitope immune profiling in different ethnic groups opens the discussion to the necessity of tuning vaccine candidates to the targeted population.

Bioinformatic tools not only help improve biosafety and coverage of new immunogens. Computational approaches allow rational designs, predicting B or T-cell epitopes with potential long-lasting protective immunity. Although the definition of correlates of protection can be complex for a multi-target vaccine and can be determined by many factors, recent vaccine candidates tend to preconize the generation of cellular over humoral response (Plotkin, Front Immunol 13, 1081107, 2022). However, the literature is vaster regarding B-cell epitopes and antibody response- and the lack of knowledge of T-cell epitopes or overall cellular response during infection can represent a challenge for a comprehensive vaccine design (Rueckert et al., PLOS Pathog 8, e1003001, 2012). Most of the epitopes selected by our pipeline could activate T-cells and promote cytokine secretion on at least one of the viruses or species tested in this work. It is important to note that, although selected as MHC-I or MHC-II specific epitopes, all the analyses were realized on PBMC pools, and no depletion of T-cell populations was performed, which could explain the dual activation of CD4⁺ or CD8⁺ cells by some epitopes, with secretion of IFN-γ, TNF-α, and IL-2. The analysis did not include other important cell lines, like natural killer (NK). Interestingly, in a trivalent MVA-based vaccine against VEEV, EEEV, and WEEV, E2 peptides were responsible for a significant IFN-gamma response without detectable immune interference seen in terms of neutralizing antibodies-which corroborates peptides MHCII-Pep1 and MHCII-Pep2 high cytokine activation in our results and highlights these epitopes as potential candidates. CD107a is commonly used as a surrogate marker for cell degranulation and cytotoxicity, and thoughtful consideration in selecting epitopes that exacerbate its secretion is advisable. However, recently, researchers demonstrated that NK cells can play a role in regulating vaccine-elicited T-cell and B-cell response (Wagstaffe et al., Clinical & Translational Immunology 7, e1010, 2018). In fact, upon vaccination with a SIV DNA/adenovirus in primates, authors observed a trend of CD107a increasing expression in NK cells stimulated in vitro, suggesting the development of memory-like NK cells and enhanced cytokine response (Vargas-Inchaustegui et al., Front Immunol 7, 340, 2016). Other studies hypothesize that NK cells can modulate the quality of the T and B cell memory responses (Cox et al., Trends in Pharmacological Sciences 42, 789-801, 2021). Similar peptide immunogenicity patterns were observed when compared to the naïve t-cell primed and expanded in vitro, with means that this approach can be used to prospect new reactive epitopes —even if biological material from infected patients is not available or from agents are restricted to biosafety levels 3 or 4.

Beyond alphaviruses, this work represents an iterative approach to vaccine design that serves the unique needs of emerging infectious disease bio-surveillance. These needs are that the workflow from identifying a target pathogen to designing a vaccine candidate is succinct and can be executed quickly. Related to this, such a workflow also needs to be easily re-executed repeatedly as more pathogenic data is collected (e.g. new biospecimens being sequenced). Also important, such a workflow needs to serve the needs of ad-hoc and exploratory analysis by storing its data in such a way that these analyses can take place on a central data source. Lastly, such a workflow needs to be constructed transparently and portable as emerging vaccine designs will take place in uniquely distributed settings with many different laboratories and researchers contributing data, performing validation assays, or conducting additional analyses. This pipeline can be executed against any collection of proteomes and implements standard workflows such as epitope identification and structural analysis. Moreover, this pipeline has a runtime of approximately 48 hours on 256 computer cores, 1000 GB of RAM, and 4× Nvidia Tesla A100 80 GB GPUs. At UTMB, these resources are available from an internal HPC cluster, but as this pipeline is entirely coordinated by Nextflow with containerization of all workflow steps, it is portable to any HPC environment and also to distributed cloud computing environments across all major vendors. Importantly, no single step requires more than a single GPU and can be executed with as little as 32 computer cores and 128 GB RAM, making this pipeline scalable through cloud pipeline engines such as AWS Batch, and Azure Batch. We hope that the pipeline presented herein can serve as a repeatable workflow for the iterative design of vaccines against emerging infections and that the broader community can use and even extend this workflow in accordance with the architecture described above.

### I. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A. Results

Comprehensive in silico analysis of MHC-I and MHC-II peptide signatures indicates overlap among alphaviruses.

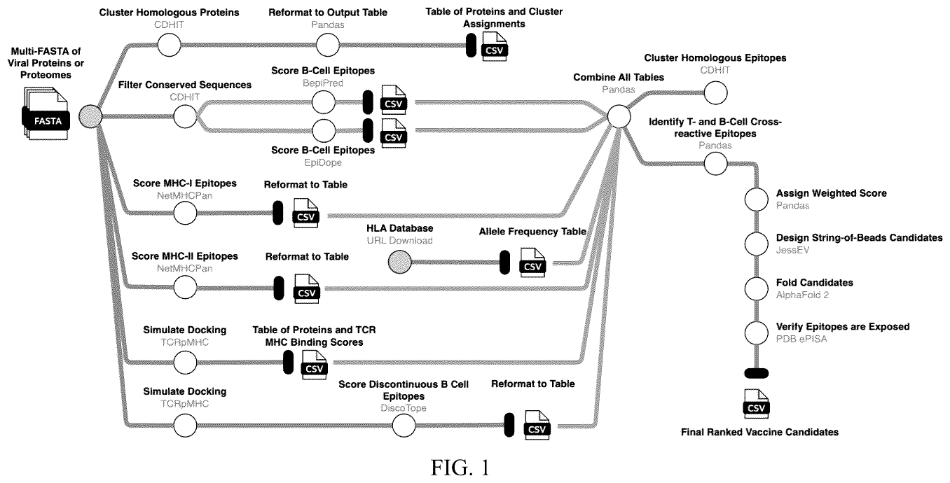

The vaccine design pipeline begins with a collection of target proteomes representing the viruses against which the vaccine is intended to be protective (Table 2). The pipeline consists of three core design phases: T-cell epitope profiling, B-cell epitope profiling, and vaccine candidate design (FIG. 1).

For B-cell profiling, the pipeline accepts a list of UniProt accessions or protein sequences that represent targets against which effective antibodies would be raised (i.e. effective targets of neutralizing antibodies). From here, the initial proteomes are BLASTed against each submitted target protein, and multiple sequence alignment is performed to identify the conserved protein sequences from among the proteomes and the target sequences. These are then submitted to epitope detection using EpiDope and BepiPred.

For T-cell profiling, the submitted proteomes undergo epitope prediction for both MHC-I and MHC-II epitopes using netMHCpan and all alleles common to the target vaccine region. Following this, epitopes whose predicted binding affinity is in the highest 5% of all scored peptides are carried forward in a model of the TCR-p-MHC complex for each epitope. Finally, free solvation energy and surface area (both derived from TCR-p-MHC models) as well as predicted binding affinity and allele frequency are combined to create a weighted immunogenicity score. Lastly, for vaccine candidate design, scored T-cell epitopes are submitted to JessEV to render vaccine candidate designs.

TABLE 2
List of accession numbers for viral proteomes
used for the epitope selection.

| Accession | Name |
|---|---|
| AB032553_1 | Getah Virus |
| AF075251_1 | Everglades Virus |
| AF075253_1 | Mucambo Virus |
| AF075254_1 | Tonate Virus |
| AF075255_1 | Venezuelan Equine Encephalitis |
| | Virus |
| AF075256_1 | Pixuna Virus |
| AF075257_1 | Mosso Pas Pedras Virus |
| AF075258_1 | Rio Negro Virus |
| AF075259_1 | Cabassou Virus |
| AF079456_1 | O'nyong-nyong Virus |
| AF079457_1 | O'nyong-nyong Virus |
| AF103728_1 | Sindbis Virus |
| AF126284_1 | Aura Virus |
| AF214040_1 | Western Equine Encephalitis Virus |
| AF237947_1 | Mayaro Virus |
| AF252265_1 | Trocara Virus |
| AF369024_2 | Chikungunya Virus |
| AF375051_1 | Venezuelan Equine Encephalitis |
| | Virus |
| AF429428_1 | Sindbis Virus |
| AJ316246_1 | Salmon Pancreas Disease Virus |
| AY702913_1 | Getah Virus |
| DQ241303_1 | Madariaga Virus |
| EF011023_1 | Getah Virus |
| EF151503_1 | Madariaga Virus |
| EF536323_1 | Middelburg Virus |
| FJ827631_1 | Highlands J Virus |
| GQ281603_1 | Fort Morgan Virus |
| GQ287646 | Western Equine Encephalitis Virus |
| GQ433354_1 | Ross River Virus |
| HM147984_1 | Sindbis Virus |
| HM147985_1 | Bebaru Virus |
| HM147986_1 | Fort Morgan Virus |
| HM147989_1 | Ndumu Virus |
| HM147990_1 | Southern Elephant Seal Virus |
| HM147991_1 | Trocara Virus |
| HM147992_1 | Una Virus |
| HM147993_1 | Whataroa Virus |
| J02363_1 | Sindbis Virus |
| JF972635_1 | Semliki Forest Virus |
| JX678730_1 | Eilat Virus |
| KJ469640_1 | Madariaga Virus |
| KM115530_1 | Middelburg Virus |
| KP003813_2 | Chikungunya Virus |
| L00930_1 | Venezuelan Equine Encephalitis |
| | Virus |
| L01442_2 | Venezuelan Equine Encephalitis |
| | Virus |
| M20162_1 | Ross River Virus |
| M20303_1 | O'nyong-nyong Virus |
| M69205_1 | Sindbis Virus |
| MK353339_2 | Caaingua Virus |
| U34999_1 | Venezuelan Equine Encephalitis |
| | Virus |
| U73745_1 | Barmah Forest Virus |
| X04129_1 | Semliki Forest Virus |
| X63135_1 | Eastern Equine Encephalitis Virus |

Peptide microarray indicates overlapping MHC-I and MHC-II epitopes among Encephalitic and Arthritogenic alphaviruses. Intending to improve the pipeline selection of the most immunogenic pan-alphavirus peptides, in vitro assessment of peptide reactivity using a slide microarray (PEPperPRINT©, Heidelberg, GER) tested against pre-exposed sera from infected human, mouse, or nonhuman primates (NHP). For this, we selected 726 epitopes, ranked by species, T-cell receptor (MHC-I or MHC-II), and presence or absence in a B-cell receptor cluster. Human samples were selected from cohorts infected with Madariaga virus (MADV), Venezuelan equine encephalitis virus (VEEV), and chikungunya virus (CHIKV). Mouse sera bank, however, included not only CHIKV and VEEV-infected samples, but also Mayaro virus (MAYV-infected) and sera from animals vaccinated with TC-83-a VEEV live attenuate vaccine commonly used as surrogate in VEEV pathogenesis studies. Finally, NHP sera bank includes CHIKV-infected samples, and a Zika virus (ZIKV) sera as an off-target control of another arbovirus (Table 3).

TABLE 3
Mouse, non-human primate, and human sera banks used
for epitope microarray reactivity analysis.

| Species | Virus | Sample ID | Additional info |
|---|---|---|---|
| Mouse | Naïve | ABSL2#56 m1 | PRNT negative TC-83/MAYV/ZIKV |
| | | ABSL2#56 m2 | PRNT negative TC-83/MAYV/ZIKV |
| | | ABSL2#56 m3 | PRNT negative TC-83/MAYV/ZIKV |
| | | ABSL2#56 m4 | PRNT negative TC-83/MAYV/ZIKV |
| | | ABSL2#56 m5 | PRNT negative TC-83/MAYV/ZIKV |
| | ZIKV | ABSL2#56 m16 | PRNT₅₀ > 1:640/PRNT₈₀ > 1:640. |
| | | ABSL2#56 m17 | PRNT₅₀ > 1:640/PRNT₈₀ > 1:640. |
| | | ABSL2#56 m18 | PRNT₅₀ > 1:640/PRNT₈₀ > 1:640. |
| | | ABSL2#56 m19 | PRNT₅₀ > 1:640/PRNT₈₀ > 1:640. |
| | | ABSL2#56 m20 | PRNT₅₀ > 1:640/PRNT₈₀ > 1:640. |
| | | ABSL2#56 m21 | PRNT₅₀ > 1:640/PRNT₈₀ > 1:640. |
| | VEEV | ABSL2#51a m29 | PRNT₅₀ > 1:640/PRNT₈₀ > 1:640 |
| | | ABSL2#51a m30 | PRNT₅₀ > 1:640/PRNT₈₀ > 1:640 |
| | | ABSL2#51b m17 | PRNT₅₀ > 1:640/PRNT₈₀ > 1:640 |
| | | ABSL2#51b m19 | PRNT₅₀ > 1:640/PRNT₈₀ > 1:640 |
| | MAYV | Study1 m17 | 36 dpi. PRNT₅₀ > 1:640/PRNT₈₀ > 1:640. |
| | | Study1 m22 | 36 dpi. PRNT₅₀ 1:40/PRNT₈₀ < 1:20. |
| | CHIKV | m1 | PRNT₅₀ > 1:640/PRNT₈₀ > 1:640 |
| | | m2 | PRNT₅₀ > 1:640/PRNT₈₀ > 1:160 |
| NHP | Naïve | Cynomolgus naïve | Pre-immune serum. |
| | ZIKV | Saimiri naïve | Pre-immune serum. |
| | | ABLS2#1912100 | 28 dpi. PRNT₅₀ 1:160/PRNT₈₀ 1:80. |
| | | NHP6550 | |
| | | ABLS2#1912100 | 28 dpi. PRNT₅₀ 1:80/PRNT₈₀ < 1:20. |
| | | NHP4806 | |
| | CHIKV | MV-CHIKV-204 pool | PRNT₅₀ > 1:640/PRNT₈₀ 1:160 |
| | | high | |
| Human | Naïve | Control serum | Millipore Sigma, REF #NIST ® SRM ® 909c |
| | | (reference sample) | |
| | VEEV* | 702193 | PRNT₈₀ 1:80 |
| | | 702196 | PRNT₈₀ 1:160 |
| | | 702218 | PRNT₈₀ 1:320 |
| | | 702228 | PRNT₈₀ 1:160 |
| | | 702229 | PRNT₈₀ 1:80 |
| | | 702241 | PRNT₈₀ 1:640 |
| | MADV* | 702216 | PRNT₈₀ 1:640 |
| | | 702226 | PRNT₈₀ 1:160 |
| | | 702231 | PRNT₈₀ 1:20 |
| | | 702232 | PRNT₈₀ 1:640 |
| | CHIKV^{#} | A02 307 | PRNT₈₀ > 1:20 |
| | | A02 537 | PRNT₈₀ > 1:20 |
| | | A02 560 | PRNT₈₀ > 1:20 |
| | | FB 307 | PRNT₈₀ > 1:20 |
| | | A01 307 | PRNT₈₀ > 1:20 |
| | | A01 54 | PRNT₈₀ > 1:20 |
| | | A01 712 | PRNT₈₀ > 1:20 |
| | | A01 560 | PRNT₈₀ > 1:20 |
| | | A01 447 | PRNT₈₀ > 1:20 |
| | | A02 48 | PRNT₈₀ > 1:20 |
| | | A02 54 | PRNT₈₀ > 1:20 |
| | | A02 712 | PRNT₈₀ > 1:20 |
| | | A02 799 | PRNT₈₀ > 1:20 |
| | | A02 961 | PRNT₈₀ > 1:20 |
| | | A02 1127 | PRNT₈₀ > 1:20 |
| | | A02 554 | PRNT₈₀ > 1:20 |

As expected, a majority of the epitopes recognize 2 or more viruses for either species tested (FIG. 2A, 2B, 2F). In an overall analysis of the most reactive epitopes, we can observe clustered areas for both human, mouse, and NHP positive samples (FIG. 7). None of the tested naïve sera presented significant hits. Unsurprisingly, both encephalitic viruses evaluated with human samples (MADV and VEEV) have the highest number of common reactive epitopes (n=533) (FIG. 2A). However, the pipeline was able to select more than 100 highly reactive epitopes (z-score>1.0) for both tested species and viruses. Among overlapping epitopes, we found 154 pan-alphavirus epitopes for humans and 257 for mice. From those, 58 epitopes were considered reactive against all virus tested on both species (FIG. 2C). Although not the main focus of pipeline, epitopes sequences that cluster on both T and B-cell receptors were identified, with epitopes crossing not only the pan-alpha spectra but also being reactive for more than one species-characterizing a good candidate for the final peptide selection (FIG. 2D-2E). It is important to reiterate that the general approach of this vaccine design pipeline is to select T-cell epitopes primarily but to make that selection such that the vaccine payload has cross-reactivity to B-cells. This is as opposed to doing extensive B-cell epitope selection and optimization. The goal, therefore, of scoring B-cell epitopes and then filtering them based upon a known list of B-cell-relevant proteins is to keep track of the specific linear and discontinuous B-cell epitopes that we want to be cross-reactive with the selected T-cell epitopes. Finally, top-ranked epitopes with high binding affinity to MHC-I (n=9) and MHC-II (n=8) receptors were selected and synthesized for in vitro validation. Majority of these peptides are included in the Envelope protein complex sequence of alphaviruses. Full description of receptor type, classification, species selection, and virus root can be found on Table 4.

TABLE 4
Final selected MHC-I and MHC-II epitopes.

| | | T-cell | B-cell | Classification | | Protein |
|---|---|---|---|---|---|---|
| ID | Sequence | type | cluster? | Species | Root | position |
| MHC-I | APLQHTAPF | MHC-I | No | top_ranked | Chikungunya virus; | E1 |
| Pep1 | (SEQ ID NO: 1) | | | human_primate_ | O'nyong-nyong virus; | |
| | | | | mouse | Igbo Ora virus | |
| MHC-I | APRRRVGGF | MHC-I | No | top_ranked | Madariaga virus; | nsp3 |
| Pep2 | (SEQ ID NO: 2) | | | human_primate_ | Eastern equine | |
| | | | | mouse | encephalitis virus | |
| MHC-I | FPSISTTAW | MHC-I | No | top_ranked | Barmah Forest virus | E1 |
| Pep3 | (SEQ ID NO: 3) | | | human_primate_ | | |
| | | | | mouse | | |
| MHC-I | HPQHHAQTF | MHC-I | No | top_ranked | Venezuelan equine | E1 |
| Pep4 | (SEQ ID NO: 4) | | | human_primate_ | encephalitis virus; | |
| | | | | mouse | Tonate virus; Mucambo | |
| | | | | | virus; Cabassou virus; | |
| | | | | | Everglades virus | |
| MHC-I | HPQLHAQTF | MHC-I | No | top_ranked | Venezuelan equine | E1 |
| Pep5 | (SEQ ID NO: 5) | | | human_primate_ | encephalitis virus; | |
| | | | | mouse | Tonate virus; Mucambo | |
| | | | | | virus; Cabassou virus; | |
| | | | | | Everglades virus | |
| MHC-I | KPDYRCQTY | MHC-I | No | top_ranked | Una virus; Semliki | E1 |
| Pep6 | (SEQ ID NO: 6) | | | human_primate_ | Forest virus; Trocara | |
| | | | | mouse | virus; Sagiyama virus; | |
| | | | | | Getah virus; Yada yada | |
| | | | | | virus; Caaingua virus | |
| MHC-I | PCCYEKGPE | MHC-I | Yes | no_mouse_no_ | Chikungunya virus; | E3 |
| Pep7 | (SEQ ID NO: 7) | | | primate_ | Mayaro virus; Ross | |
| | | | | no_human | River virus; O'nyong- | |
| | | | | | nyong virus; Igbo Ora | |
| | | | | | virus | |
| MHC-I | PCCYEKQPE | MHC-I | Yes | no_mouse_no_ | Chikungunya virus; | E3 |
| Pep8 | (SEQ ID NO: 8) | | | primate_ | Ross River virus; | |
| | | | | no_human | O'nyong-nyong virus; | |
| | | | | | Sagiyama virus; Getah | |
| | | | | | virus | |
| MHC-I | PDDQDTGSE | MHC-I | Yes | no_mouse_no_ | Sleeping disease virus; | nsp4 |
| Pep9 | (SEQ ID NO: 9) | | | primate_ | Salmonid alphavirus | |
| | | | | no_human | subtype 3 | |
| MHC-II | APCSLVSYHGY | MHC-II | Yes | human_top_ | Rio Negro virus; | E2 |
| Pep1 | YILA (SEQ ID | | | mouse_bottom | Venezuelan equine | |
| | NO: 10) | | | | encephalitis virus, | |
| | | | | | Pixuna virus; | |
| | | | | | Madariaga virus; | |
| | | | | | Eastern equine | |
| | | | | | encephalitis virus | |
| MHC-II | CYMFATARRK | MHC-II | No | top_ranked_ | Bebaru virus; Una | E2 |
| Pep2 | CLTPY (SEQ ID | | | human_mouse | virus; Chikungunya | |
| | NO: 11) | | | | virus; Middelburg virus; | |
| | | | | | Mayaro virus; Semliki | |
| | | | | | Forest virus; Western | |
| | | | | | equine encephalitis | |
| | | | | | virus | |
| MHC-II | HAGYIRIQTSA | MHC-II | No | top_ranked_ | Venezuelan equine | E2 |
| Pep3 | MFGL (SEQ ID | | | human_mouse | encephalitis virus; | |
| | NO: 12) | | | | Tonate virus; Mucambo | |
| | | | | | virus; Cabassou virus; | |
| | | | | | Madariaga virus; | |
| | | | | | Eastern equine | |
| | | | | | encephalitis virus | |
| MHC-II | IIFVNMRTPYK | MHC-II | No | top_ranked_ | Venezuelan equine | nsp2 |
| Pep4 | HHHY (SEQ ID | | | human_mouse | encephalitis virus, | |
| | NO: 13) | | | | Madariaga virus; | |
| | | | | | Eastern equine | |
| | | | | | encephalitis virus | |
| MHC-II | KALITQRMLK | MHC-II | No | top_ranked_ | Rio Negro virus; | nsp4 |
| Pep5 | GLGHY (SEQ ID | | | human_mouse | Venezuelan equine | |
| | NO: 14) | | | | encephalitis virus; | |
| | | | | | Pixuna virus, | |
| | | | | | Madariaga virus; | |
| | | | | | Eastern equine | |
| | | | | | encephalitis virus | |
| MHC-II | KLFLAKSATRS | MHC-II | No | top_ranked_ | Bebaru virus | nsp2 |
| Pep6 | IVER (SEQ ID | | | human mouse | | |
| | NO: 15) | | | | | |
| MHC-II | LARRFSSFRAV | MHC-II | No | top_ranked_ | Mayaro virus; Ross | E2 |
| Pep7 | TVRC (SEQ ID | | | human_mouse | River virus; Sagiyama | |
| | NO: 16) | | | | virus; Getah virus | |
| MHC-II | LASCYMFATA | MHC-II | No | top_ranked_ | Una virus; Middelburg | E2 |
| Pep8 | RRKCL (SEQ ID | | | human_mouse | virus; Mayaro virus; | |
| | NO: 17) | | | | Semliki Forest virus; | |
| | | | | | Ross River virus; | |
| | | | | | Sagiyama virus; Getah | |
| | | | | | virus | |

Molecular dynamics (MD) simulations agree with in silico and in vitro predictions from the pipeline. To assess the feasibility of the identified peptides to recognize and bind MHC- or MHC-II molecules in a structure-based context, we employed molecular modeling and molecular dynamics (MD) simulations approaches. Initially, we modeled the peptides into two prevalent HLAs: HLA A*02:01:01:01 for MHC-I and HLA-DRA1: DRB1*07:01:01:01 for MHC-II, utilizing two AlphaFold-based approaches: a customized version of AlphaFold2-multimer (AF2M) version and ColabFold. Subsequently, we performed MD simulations to evaluate the stability of the peptide in the MHC binding site. For the MHC-I peptides, 7 out of 9 identified peptides (MHCI-pep1, 2, 3, 4, 5, 6 and 9) were successfully modeled by AF2M or ColabFold, exhibiting a canonical binding mode, as assessed by estimating the deviation from known peptide-bound 3D structures (Table 5). These 7 peptides displayed appropriate anchors at the P2 and PQ MHC-I binding sites. The remaining 2 peptides (MHCI-pep7 and MHCI-pep8) did not exhibit correct placement of the peptide C-terminal to the MHC-I PQ binding site in our models. In addition to the proper binding mode, MHCI-pep1, MHCI-pep4 and MHCI-pep5 achieved high confidence modeling scores (>0.85) (Table 5).

TABLE 5
Analysis of peptide models bound to MHC generated
by AlphaFold2-Multimer (AF2M) or ColabFold.

| | | | Comparative peptide | |
|---|---|---|---|---|
| | | | backbone RMSD (Å)/PDB | Confidence |
| MHC | MHC alelle | peptide | reference | score |
| Class I | A*02 | pep1 | 1.8/5eu4 (AF2M) | 0.92 (AF2M) |
| | | | 1.8/5eu4 (ColabFold) | 0.92 (ColabFold) |
| | | pep2 | 4.1/2gtw (AF2M) | 0.68 (AF2M) |
| | | | 0.8/5eu4 (ColabFold) | 0.79 (ColabFold) |
| | | pep3 | 2.1/2gtw (AF2M) | 0.60 (AF2M) |
| | | | 1.7/2gtw (ColabFold) | 0.6 (ColabFold) |
| | | pep4 | 0.6/3 ft4 (AF2M) | 0.91 (AF2M) |
| | | | 0.6/5eu5 (ColabFold) | 0.87 (ColabFold) |
| | | pep5 | 0.6/3 ft4 (AF2M) | 0.91 (AF2M) |
| | | | 0.6/5hhn (ColabFold) | 0.88 (ColabFold) |
| | | pep6 | 0.6/5hhn (AF2M) | 0.74 (AF2M) |
| | | | 0.6/6ptb (ColabFold) | 0.66 (ColabFold) |
| | | pep7 | 13.7/2gtw (AF2M) | 0.85 (AF2M) |
| | | | 1.9/2x4s (ColabFold) | 0.68 (ColabFold) |
| | | pep8 | 2.6/2v2x (AF2M) | 0.75 (AF2M) |
| | | | 2.23/2v2x (ColabFold) | 0.73 (ColabFold) |
| | | pep9 | 1.2/7lg2 (AF2M) | 0.74 (AF2M) |
| | | | 1.9/1qr1 (ColabFold) | 0.68 (ColabFold) |
| Class II | DRA1:DRB1*07:01:01:01 | pep1 | 1.2/3l6f (AF2M) | 0.91 (AF2M) |
| | | | 2.4/3l6f (ColabFold) | 0.87 (ColabFold) |
| | | pep2 | 2.1/1klg (AF2M) | 0.89 (AF2M) |
| | | | 1.7/4z7u (ColabFold) | 0.87 (ColabFold) |
| | | pep3 | 1.8/6blx (AF2M) | 0.87 (AF2M) |
| | | | 2.3/6blx (ColabFold) | 0.85 (ColabFold) |
| | | pep4 | 4.2/4z7u (AF2M) | 0.86 (AF2M) |
| | | | 3.8/1sje (ColabFold) | 0.85 (ColabFold) |
| | | pep5 | 1.3/2ian (AF2M) | 0.87 (AF2M) |
| | | | 1.1/2ian (ColabFold) | 0.88 (ColabFold) |
| | | pep6 | 2.9/3cup (AF2M) | 0.87 (AF2M) |
| | | | 1.3/2ian (ColabFold) | 0.92 (ColabFold) |
| | | pep7 | 1.5/6blx (AF2M) | 0.92 (AF2M) |
| | | | 1.5/6blx (ColabFold) | 0.90 (ColabFold) |
| | | pep8 | 6.1/6blx (AF2M) | 0.85 (AF2M) |
| | | | 10.7/6blx (ColabFold) | 0.87 (ColabFold) |

Peptides were molded bound to the corresponding MHC alleles (A*02 or DRA1:DRB1*07:01:01:01). The feasibility of modeled binding was assessed by comparing peptide backbone structures to a reference set of 3D structures of peptides bound to MHC-I or MHC-II molecules. The table includes the lowest observed deviation, assessed by the root-mean-square deviation (RMSD), along with the corresponding PDB reference structure. Additionally, confidence scores provided by AF2M or ColabFold are listed. This score corresponds to the combination of ipTM and pTM scores (0.8*ipTM + 0.2*pTM), as described in (1). For MHC-II molecules, the provided AF2M or ColabFold scores incorporate the interface between the MHC chains in the calculation by default.

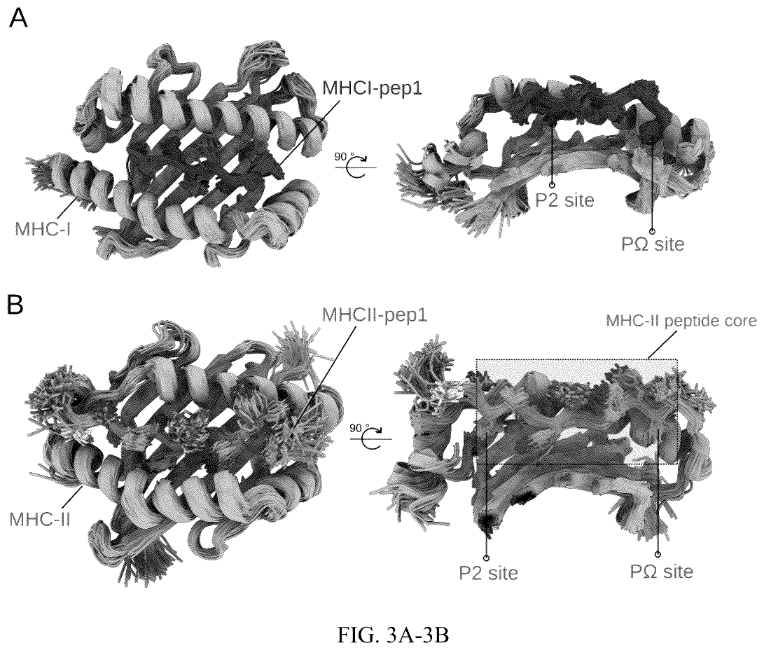

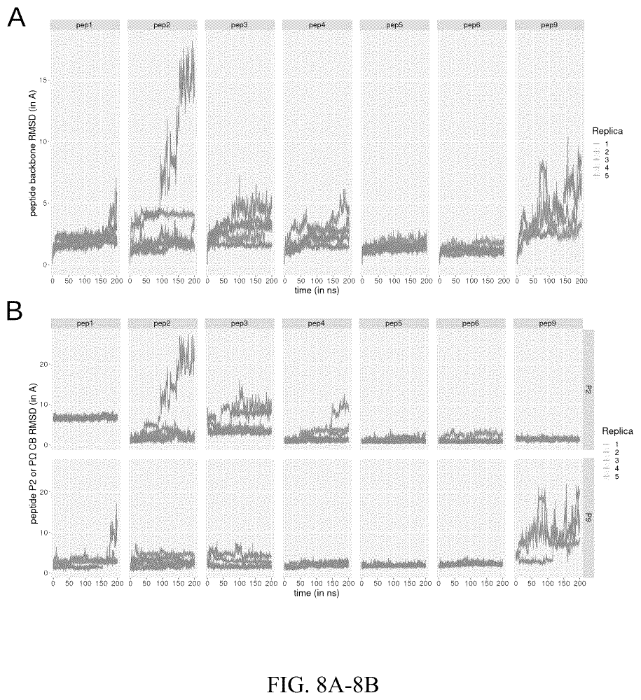

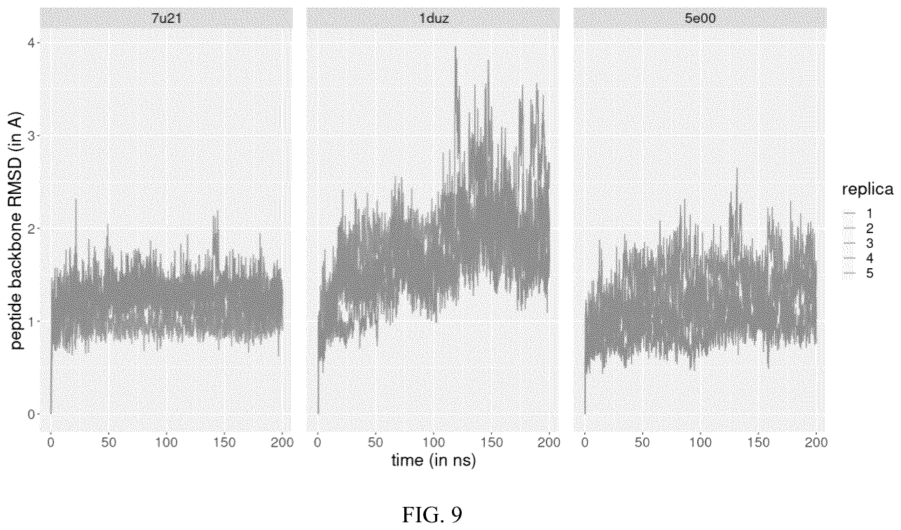

Through 200 ns MD simulations across 5 independent replicas, initiated from the modeled complexes, we observed that the 7 peptides with a canonical binding mode were generally stable, maintaining the MHC-I anchored sites in most replicas. Notably, MHCI-pep1, MHCI-pep5 and MHCI-pep6 exhibited the lowest deviation along the trajectory (median peptide backbone RMSD of 2.11, 1.48 and 1.17, respectively, considering the last half of the trajectory) in all evaluated replicas (FIG. 8), consistent with the deviation observed in known binder peptides (FIG. 9). As a representative case, FIG. 3 illustrates the ensemble of MHCI-pep1 conformations obtained from the simulated trajectory.

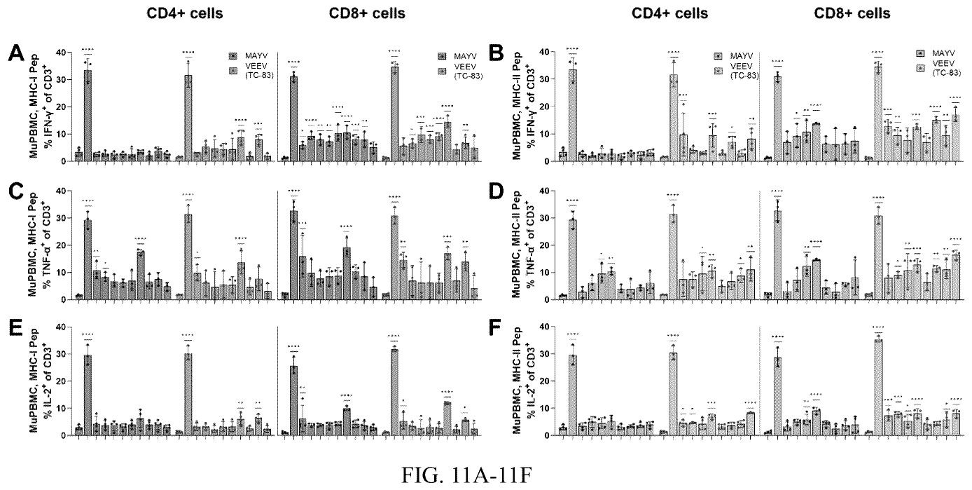

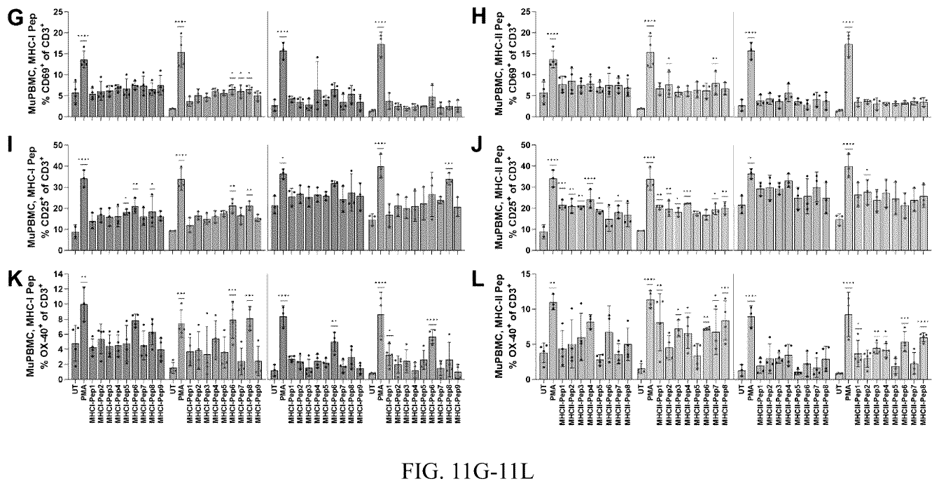

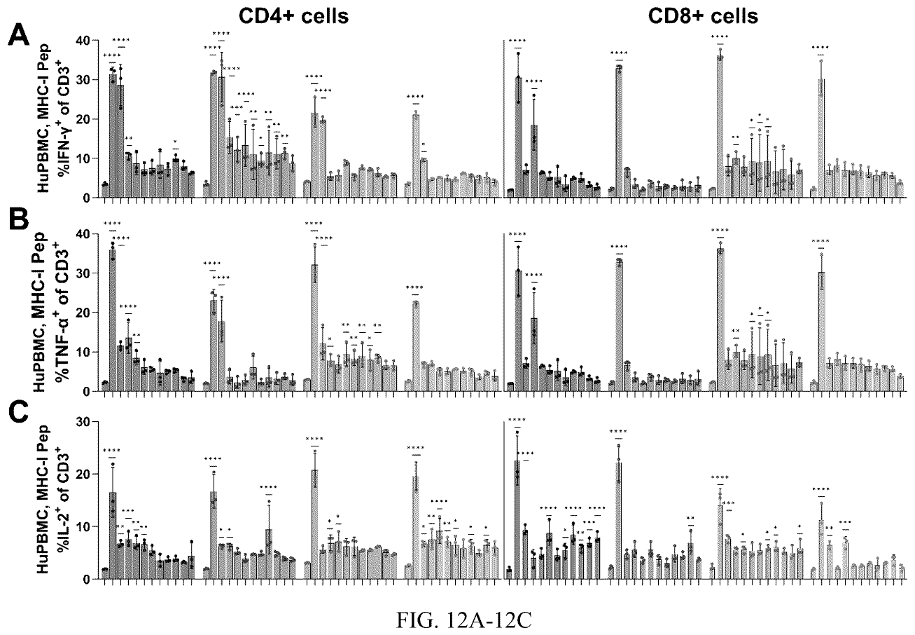

For the MHC-II peptides, 6 out of 8 peptides were successfully modeled by AF2M or ColabFold with a canonical binding mode (Table 5). Two peptides (MHCII-pep4 and MHCII-pep8) did not exhibit appropriate fitting of the C-terminal into the MHC-II groove in our models. Since modeling peptides bound to MHC-II is typically more challenging than MHC-I cases due to their increased susceptibility to positional shifting, we employed an orthogonal method and compared the peptide binding core predicted by NetMHCIIpan with the core observed in the 3D models. NetMHCIIpan predictions corroborated the binding core of 4 peptides: MHCII-pep1, MHCII-pep5, MHCII-pep6 and MHCII-pep7, thereby supporting confidence in the models (Table 6). All 6 modeled peptides that exhibited canonical binding modes also demonstrated high backbone stability at the peptide core in the MD simulations (FIG. 11). As a representative case, FIG. 3 illustrates the ensemble of the MHCII-pep1 conformations obtained from the simulated trajectory. Taking together, these structure-based in silico results suggest the feasibility of most peptides identified in the pipeline to correctly bind to MHC-I and MHC-II alleles, assuming canonical binding modes. Furthermore, the MD simulations indicated that the majority of peptides stably bind to the MHC grooves, maintaining the anchored peptide positions at MHC binding sites.

TABLE 6
Identification and prediction of the binding
cores of the MHC-II bound peptides.

| | | NetMHCIIpan |
|---|---|---|
| | Peptide core | core prediction/ |
| Peptide | in 3D model | reliability score |
| MHCII_pep1 | APCSLVSYHGYYILA | APCSLVSYHGYYILA/ |
| | (SEQ ID NO: 10) | 0.972 (SEQ ID NO: 10) |
| MHCII_pep2 | CYMFATARRKCLTPY | CYMFATARRKCLTPY/ |
| | (SEQ ID NO: 11) | 0.533 (SEQ ID NO: 11) |
| MHCII_pep3 | HAGYIRIQTSAMFGL | HAGYIRIQTSAMFGL/ |
| | (SEQ ID NO: 12) | 0.767 (SEQ ID NO: 12) |
| MHCII_pep4 | NA | IIFVNMRTPYKHHHY/ |
| | | 0.593 (SEQ ID NO: 13) |
| MHCII_pep5 | KALITQRMLKGLGHY | KALITQRMLKGLGHY/ |
| | (SEQ ID NO: 14) | 0.407 (SEQ ID NO: 14) |
| MHCII pep6 | KLFLAKSATRSIVER | KLFLAKSATRSIVER/ |
| | (SEQ ID NO: 15) | 0.98 (SEQ ID NO: 15) |
| MHCII_pep7 | LARRFSSFRAVTVRC | LARRFSSFRAVTVRC/ |
| | (SEQ ID NO: 16) | 1.0 (SEQ ID NO: 16) |
| MHCII_pep8 | NA | LASCYMFATARRKCL/ |
| | | 0.927 (SEQ ID NO: 17) |

The peptide binding cores are highlighted in red. The peptide core in the 3D models generated by AF2M or ColabFold was assessed by visual inspection of MHC-II anchor sites.
NA (‘not applicable’) indicates peptides that could not be corrected modeled.
Alongside the core sequence predicted by NetMHCIIpan, the reliability score of the binding core, expressed as the fraction of networks in the ensemble (2), is also presented.

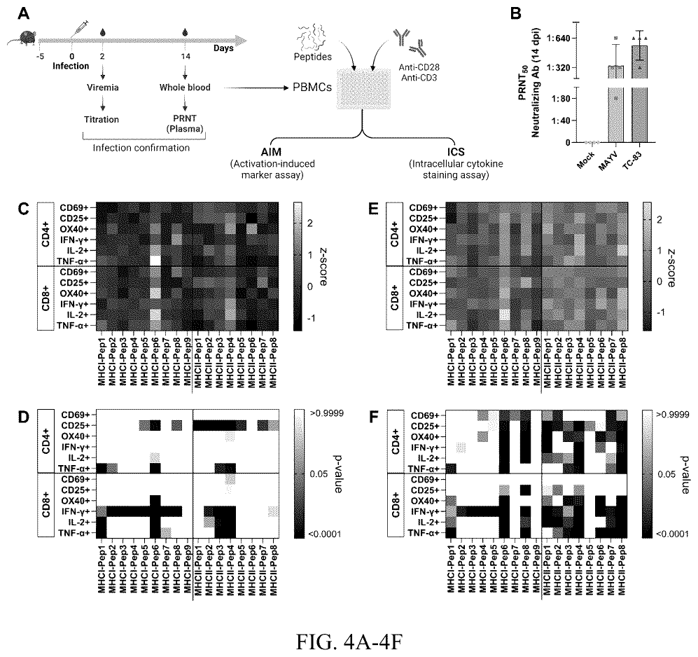

Immunogenicity profile of pre-exposed murine PBMCs re-stimulated with peptides indicates a strong T-cell activation and IFN-gamma secretion. To validate the selected peptides by T-cell antigen-specific immunogenic response, we infected mice with MAYV-a representative of the arthritogenic alphaviruses, or with TC-83-a representative of the encephalitic alphaviruses. Later, the isolated PBMC was re-stimulated ex vivo. We detected activated T-cell and effector cytokine formation by flow cytometry (FIG. 4A). Successful infection and promotion of immune response against the viruses were confirmed by neutralization assay (PRNT50, FIG. 4B). We selected surface activation-induced markers (AIM) that indicate early (CD69), moderate late (CD25), and late T-cell activation (OX-40). To further characterize the functionality of the activated T-cells, we measured the induction cytokines central to the process of T-cell priming, proliferation, recruitment, and regulation-such as IFN-gamma, TNF-alpha, and IL-2.

We can observe that both sets of peptides, with binding affinity to MHC-I or MHC-II, were able to stimulate T-cell activation. However, the MAYV response (FIG. 4C-4D) focused more on the CD8+ cells secreting IFN-gamma and activated CD4+ cells (CD25+). For MAYV reactivity, peptides MHC-I Pep1, MHC-I Pep 6, MHC-II Pep 3, and MHC-II Pep 4 promoted synergy of responses on activated TCD8-cells secreting IFN-gamma and IL-2 and activated TCD4-cells secreting TNF-alpha. In the case of TC-83 immunogenicity, all MCH-II peptides (apart from MHC-II Pep5) presented stimulation statistically significant compared with the untreated control-especially on the mid and late activation markers (CD25 and OX-40, respectively). MHC-I peptides' responses mainly agree with the MAYV results-promoting a strong IFN-gamma on TCD8 cells. Complete data plotted by positive-cell percentage can be observed in FIG. 11, including experimental controls. It is important to note that the most reactive peptides (MHC-I Pep1, MHC-I Pep 6, MHC-II Pep 3, and MHC-II Pep 4) are both identified in the pipeline as top-ranked for human and mouse MHC alleles (Table 4).

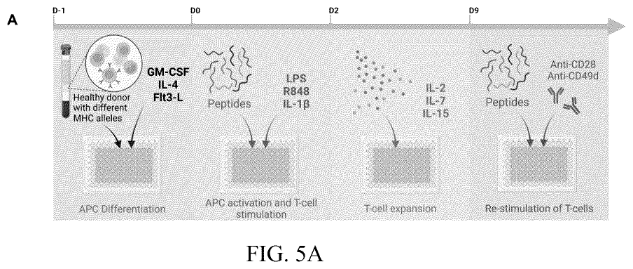

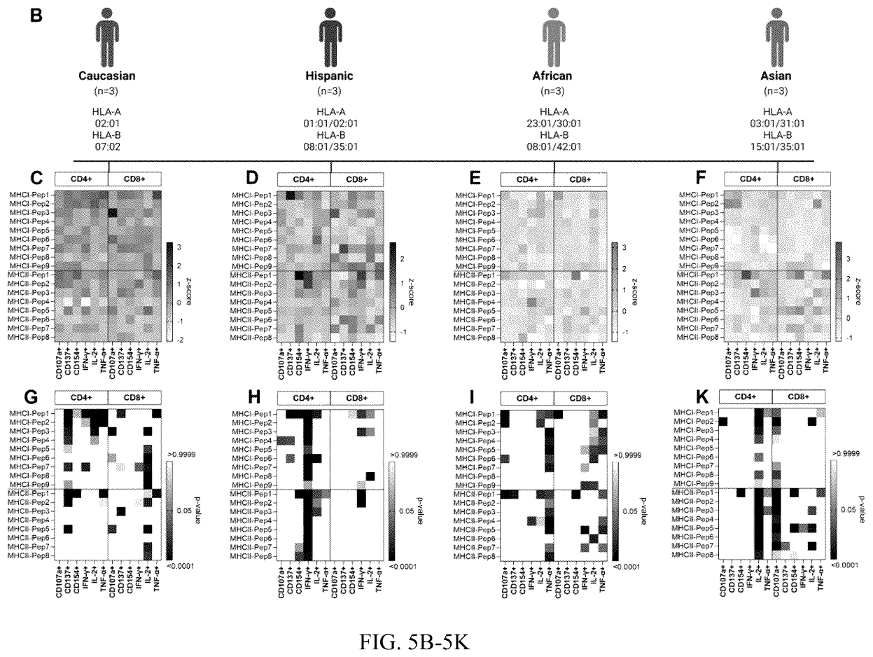

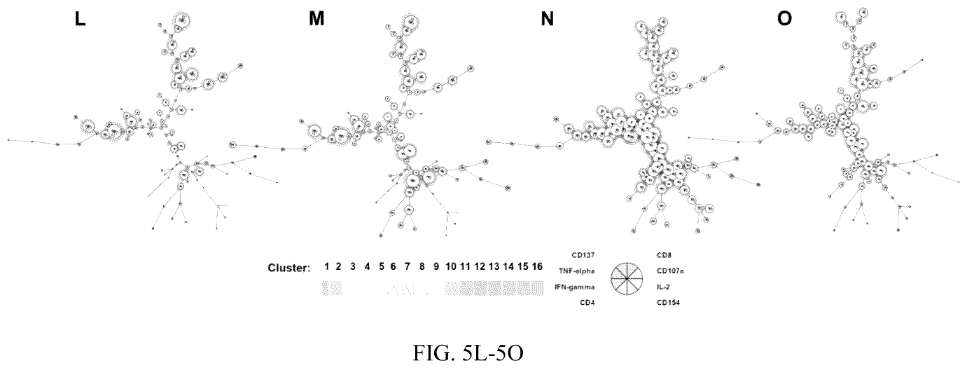

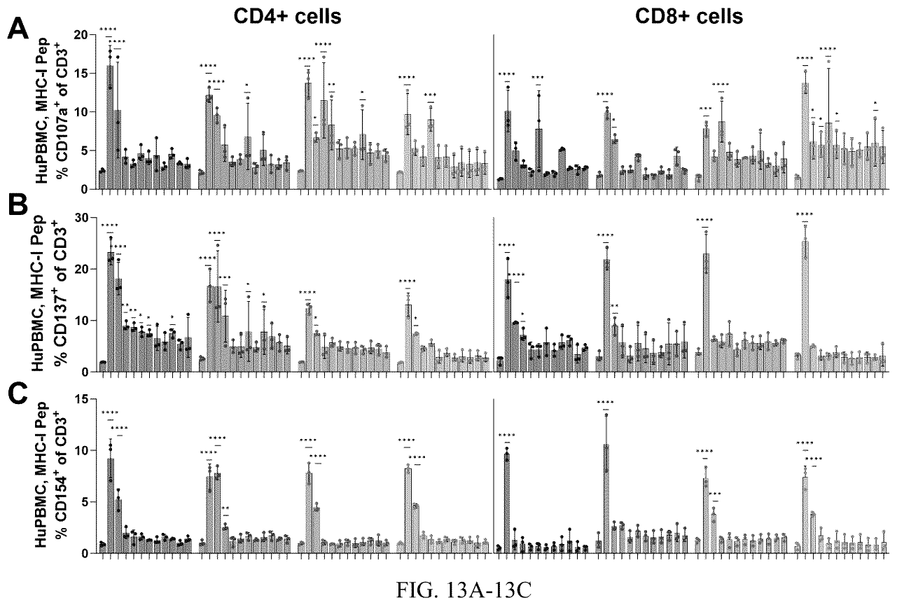

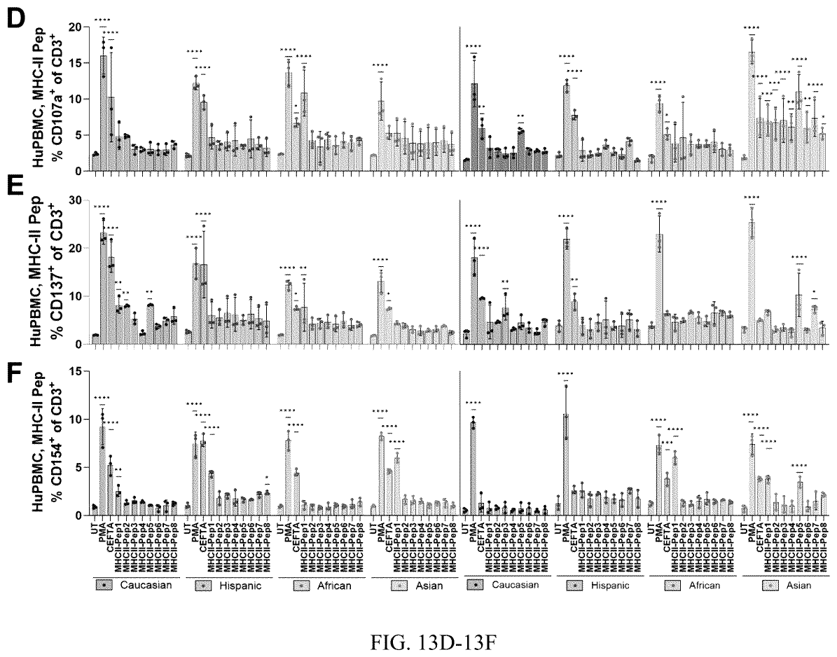

Pan-alphavirus peptides are highly immunogenic, but present different T-cell activation patterns depending on HLA allele groups. One of the main focuses of the pipeline selection was to tailor the vaccine candidate to the realities where the infectious agent circulates. Due to that, we included the most frequent HLA alleles observed in South America as a pipeline constraint, as described by molecular dynamics. To evaluate the overall impact on T-cell immunity and allele variability, we obtained HLA-typed PBMCs (ePBMC, ImmunoSpot, USA), selecting the most common HLA alleles on the ethnic groups Caucasian, Hispanic, African, and Asian. Donors' descriptives can be found in Table 7. Regarding surface markers, we included CD107a as a de-granulation marker associated with NK cell functional activity. Also, CD137 is a costimulatory protein member of the TNFR family that promotes the proliferation and survival of activated T-cells. CD154 (or CD40L), another member of the TNFR family, can correlate with T-cell activation-mainly expressed in activated CD4+ cells. (FIG. 5 and FIG. 13).

To assess the potential immunogenicity of our 17-predicted peptides, we induced T-cell-specific responses against each peptide using an immunogenicity assay designed to rapidly prime naïve T-cells (FIG. 5A). Overall, our data shows that several peptides have well-defined immunogenic signatures, leading towards a strong IFN-gamma response and moderate IL-2 secretion, with some effectively triggering TNF-alpha (FIG. 5). It is not surprising that peptides tend to be more responsive to Hispanic and African donors, especially concerning cytokines induction (FIG. 5D/5H and 5E/5I, respectively), which is expected due to the genetic background of the South American population added to the pipeline. Both sets of peptides (MHC-I and MHC-II) stimulates strong TCD4-driven IFN-gamma response in Hispanic donors, but TCD8 cell IFN-gamma reactivity is only for few MHC-II peptides. African donors have some well-balanced responses among biomarkers, presenting good candidates for the final selection. On time, even with a strong TNF-alpha response elicited by in vitro stimulated African PBMCs, we couldn't observe a correspondent reactivity with the surface markers from the same protein family (CD154 and CD137). In general, Caucasian donors promote a milder response regarding cytokines and activation markers in comparison to Hispanics, but the responsiveness of MHC-I Pep1 and MHC-II Pep1 is sustained between the majority of receptors and cytokines tested. Surprisingly, stimulated Asian PBMCs mainly elicited an IL-2-driven response by both sets of epitopes analyzed.

TABLE 7
PMBC healthy donors' descriptives, including ethnicity, age, gender, and HLA-typing. Descriptives were provided by vendor
(ImmunoSpot, USA).

| | Donor # | 1 | 2 | 3 | 4 | 5 | 6 |
|---|---|---|---|---|---|---|---|
| Demo- | Sample | HHU202206 | HHU202102 | HHU202112 | HHU201912 | HHU202003 | HRU202004 |
| graphics | ID # | 02 | 02 | 21 | 12 | 05 | 28 |
| | Collection | Jun. 1, 2022 | Feb. 1, 2021 | Dec. 20, 2021 | Jun. 29, 2020 | Mar. 4, 2020 | Apr. 28, 2020 |
| | Date | | | | | | |
| | Ethnicity | Caucasian | Caucasian | Caucasian | Hispanic | Hispanic | Hispanic |
| | Age | 38 | 31 | 36 | 39 | 54 | 51 |
| | Gender | Female | Female | Female | Male | Male | Male |
| | ABO/Rh | B/Pos | 0/Neg | 0/Pos | A/Pos | 0/Neg | 0/Pos |
| HLA | HLA-A | A*02:01/ | A*02:01/ | A*02:01/ | A*01:01/ | A*02:01/ | A*02:01/ |
| Class | | A*24:02 | A*03:01 | A*03:01 | A*68:01 | A*26:01 | A*24:03 |
| I | HLA-B | B*07:02/ | B*07:02/ | B*07:02/ | B*08:01/ | B*14:01/ | B*35:01/ |
| | | B*15:01 | B*44:02 | B*57:01 | B*15:40 | B*35:01 | B*35:12 |
| | HLA-C | C*03:03/ | C*05:01/ | C*06:02/ | C*03:03/ | C*04:01/ | C*04:01/ |
| | | C*07:02 | C*07:02 | C*07:02 | C*07:01 | C*08:02 | C*04:01 |
| HEA | HLA- | DRB1*04:01/ | DRB1*04:01/ | DRB1*07:01/ | DRB1*03:01/ | DRB1*07:01/ | DRB1*08:02/ |
| Class | DRB1 | DRB1*04:04 | DRB1*15:01 | DRB1*15:01 | DRB1*08:02 | DRB1*16:02 | DRB1*13:01 |
| II | HLA- | DQB1*03:02/ | DQB1*03:01/ | DQB1*03:03/ | DQB1*02.01/ | DQB1*02:02/ | DQB1*04:02/ |
| | DQB1 | ~ | DQB1*06:02 | DQB1*06:02 | DQB1*04:02 | DQB1*03:01 | DQB1*06:03 |
| | HLA- | DPB1*04:01/ | DPB1*04:01/ | DPB1*04:01/ | DPB1*04:01/ | DPB1*02:01/ | DPB1*04:02/ |
| | DPB1 | DPB1*05:01 | DPB1*11:01 | ~ | DPB1*105:01 | G/ | DPB1*19:01 |
| | | | | | | DPB1*04:02 | |
| | | | | | | G | |
| | HLA- | DQA1*02:01/ | DQA1*01:02/ | DQA1*01:02/ | DQA1*04:01/ | not tested | DQA1*01:03/ |
| | DQA1 | ~ | DQA1*03:01 | DQA1*02:01 | DQA1*05:01 | | DQA1*04:01 |
| | HLA- | DRB4*01:01/ | DRB4*01:01/ | DRB4*01:01/ | DRB3*01:01/ | DRB4*01:01/ | DRB3*02:02/ |
| | DRB3/4/ | ~ | DRB5*01:01 | DRB5*01:01 | ~ | DRB5*02:02 | ~ |
| | 5 | | | | | | |
| | HLA- | DPA1*01:03/ | DPA1*01:03/ | DPA1*01:03/ | DPA1*01:03/ | DPA1*01:03/ | DPA1*01:03/ |
| | DPA1 | DPA1*02:02 | DPA1*02:01 | ~ | DPA1*01:03 | DPA1*01:03 | DPA1*02:07 |
| CD16-V212F | | not tested | not tested | not tested | CD16- | CD16- | CD16- |
| | | | | | Phe/Phe | Val/Phe | Val/Phe |

PMBC healthy donors′ descriptives, including ethnicity, age, gender, and HLA-typing. Descriptives were provided by vendor
(ImmunoSpot, USA).

| | Donor # | 7 | 8 | 9 | 10 | 11 | 12 |
|---|---|---|---|---|---|---|---|
| Demo- | Sample | HHU202005 | HHU202002 | HHU202208 | HHU202108 | HHU202110 | HHU202307 |
| graphics | ID # | 07 | 13 | 25 | 33 | 07 | 27 |
| | Collection | May 6, 2020 | Feb. 12, 2020 | Mar. 6, 2023 | Aug. 20, 2021 | Oct. 6, 2021 | Jul. 25, 2023 |
| | Date | | | | | | |
| | Ethnicity | African/ | African/ | African/ | Asian | Asian | Asian |
| | | American | American | American | | | |
| | Age | 30 | 58 | 48 | 21 | 22 | 34 |
| | Gender | Male | Małe | Male | Male | Male | Female |
| | ABO/Rh | A/Pos | 0/Pos | 0/Pos | A/Pos | A/Pos | B/Pos |
| HLA | HLA-A | A*23:01/ | A*30:01/ | A*30:02/ | A*31:01/ | A*01:01/ | A*03:01/ |
| Class | | A*33:01 | A*30:01 | A*33:03 | — | A*32:01 | A*24:30 |
| I | HLA-B | B*07:06/ | B*42:01/ | B*08:01/ | B*15:01/ | B*35:01/ | B*38:02/ |
| | | B*42:01 | B*42:01 | B*58:01 | B*44:03 | B*40:06 | B*57:01 |
| | HLA-C | C*07:02/ | C*17:01/ | C*07:01/ | C*03:04/ | C*04:01/ | C*06:02/ |
| | | C*17:01 | C*17:01 | C*07:18 | C*14:03 | C*15:02 | C*07:02 |
| HEA | HLA- | DRB1*01:01/ | DRB1*03:02/ | DRB1*03:01/ | DRB1*11:01/ | DRB1*15:02/ | DRB1*07:01/ |
| Class | DRB1 | DRB1*03:02 | DRB1*11:02 | DRB1*15:03 | DRB1*13:02 | DRB1*16:02 | DRB1*12:02 |
| II | HLA- | DQB1*04:02/ | DQB1*03:19/ | DQB1*02:01/ | DQB1*02:01/ | DQB1*05:02/ | DQB1*03:03/ |
| | DQB1 | DQB1*05:01 | DQB1*04:02 | DQB1*06:02 | DQB1*06:04 | DQB1*06:01 | DQB1*05:02 |
| | HLA- | DPB1*01:01/ | DPB1*01:01 | DPB1*02:01/ | DPB1*02:01/ | DPB1*02:01/ | DPB1*02:01/ |
| | DPB1 | DPB1*85:01 | G/ | DPB1*02:01 | DPB1*04:01 | DPB1*04:01 | DPB1*21:01 |
| | | | DPB1*85:01 | | | | |
| | | | G | | | | |
| | HLA- | DQA1*01:01/ | not tested | DQA1*01:02/ | DQA1*01:02/ | DQA1*01:02/ | DQA1*02:02/ |
| | DQA1 | DQA1*04:01 | | DQA1*05:01 | DQA1*05:01 | DQA1*01:03 | DQA1*02:01 |
| | HLA- | DRB3*01:01/ | DRB3*01:01/ | DRB5*02:02/ | DRB3*02:02/ | DRB5*01:02/ | DRB3*03:01/ |
| | DRB3/4/ | ~ | DRB3*02:02 | DRB5*01:01 | DRB3*03:01 | DRB5*02:02 | DRB4*01:03 |
| | 5 | | | | | | |
| | HLA- | DPA1*02:01/ | DPA1*02:02/ | DPA1*01:03/ | DPA1*01:03/ | DPA1*01:03/ | DPA1*01:03/ |
| | DPA1 | DPA1*02:12 | DPA1*02:12 | DPA1*01:03 | ~ | ~ | DPA1*01:03 |
| CD16-V212F | | CD16- | CD16- | not tested | not tested | not tested | not tested |
| | | Phe/Phe | Val/Val | | | | |

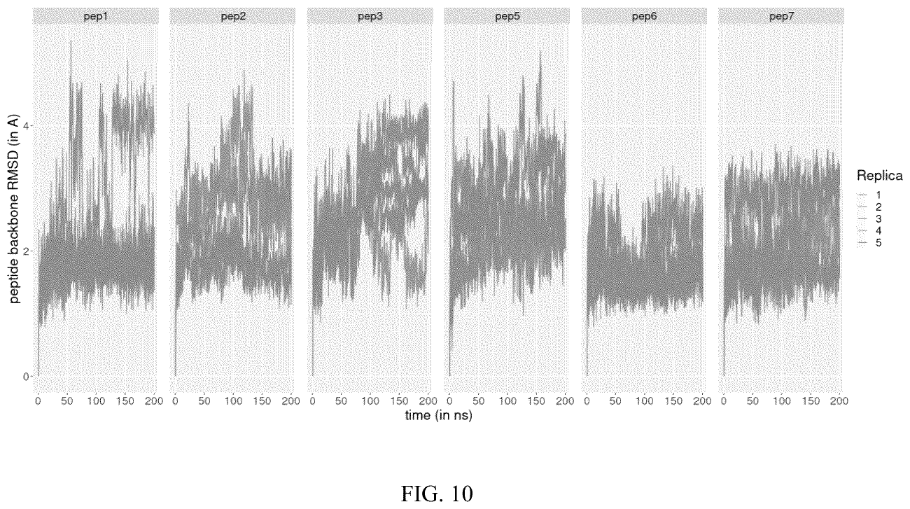

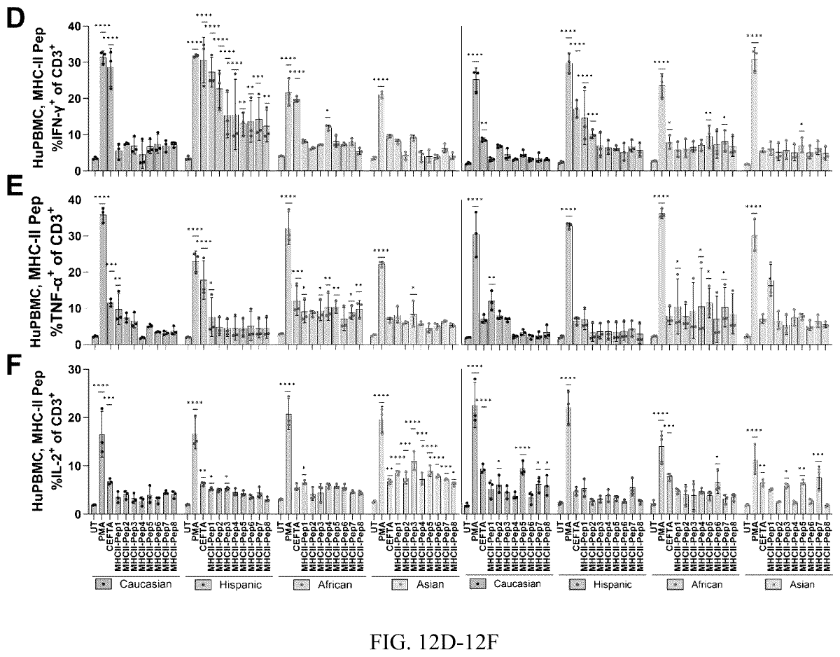

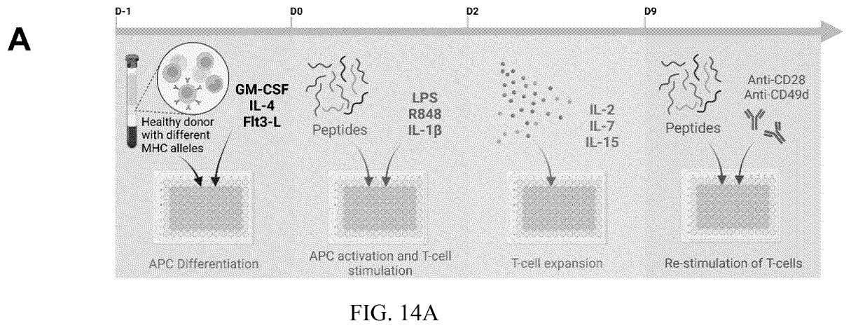

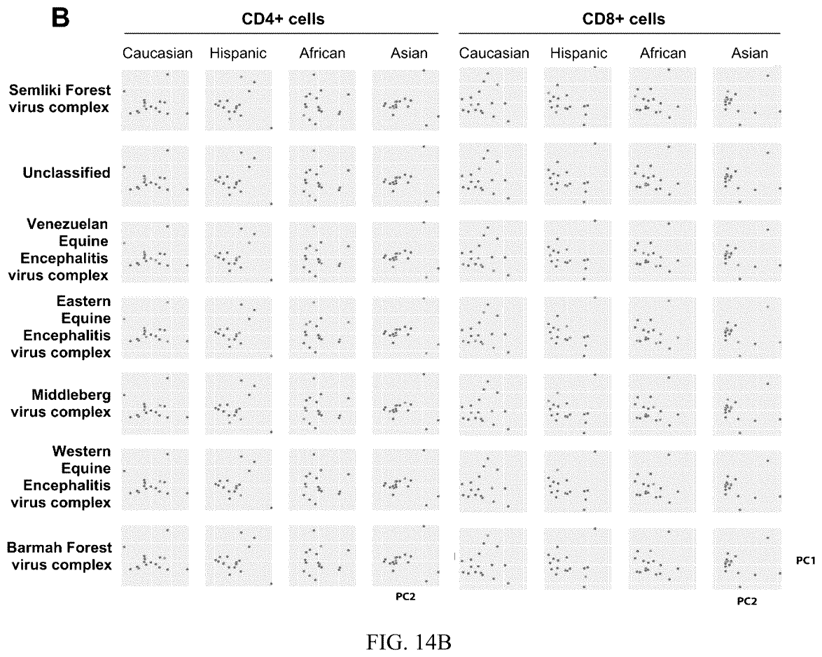

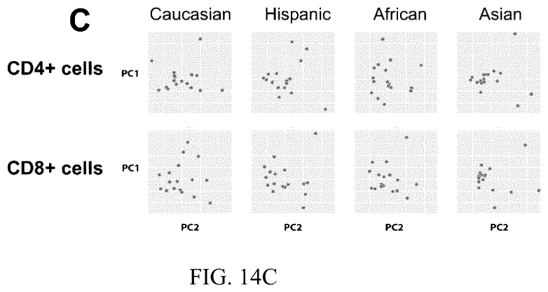

When analyzing the pattern of cellular response by ethnic group, majority of cells cluster the same way among groups but with different biomarkers intensity (FIG. 5L-50). In this analysis, it is clear how the response is milder throughout all cells analyzed on Caucasian donors, clear opposite to African response. Finally, as observed on the murine PBMC, peptide immunogenicity in humans presents different patterns when analyzed by viruses (FIG. 14B) and by ethnicity (FIG. 14C). These data show that host immune response to these peptides varies broadly not only by viral complex but also by ethnicity which illustrates a core challenge of producing a peptide vaccine with broad viral and ethnic coverage. Total stimulated cells percentage can be observed in FIGS. 12 and 13, including experimental controls.

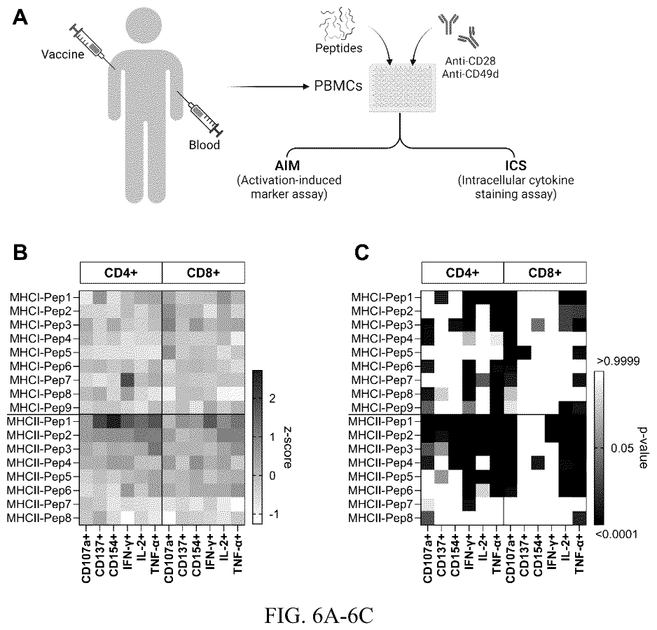

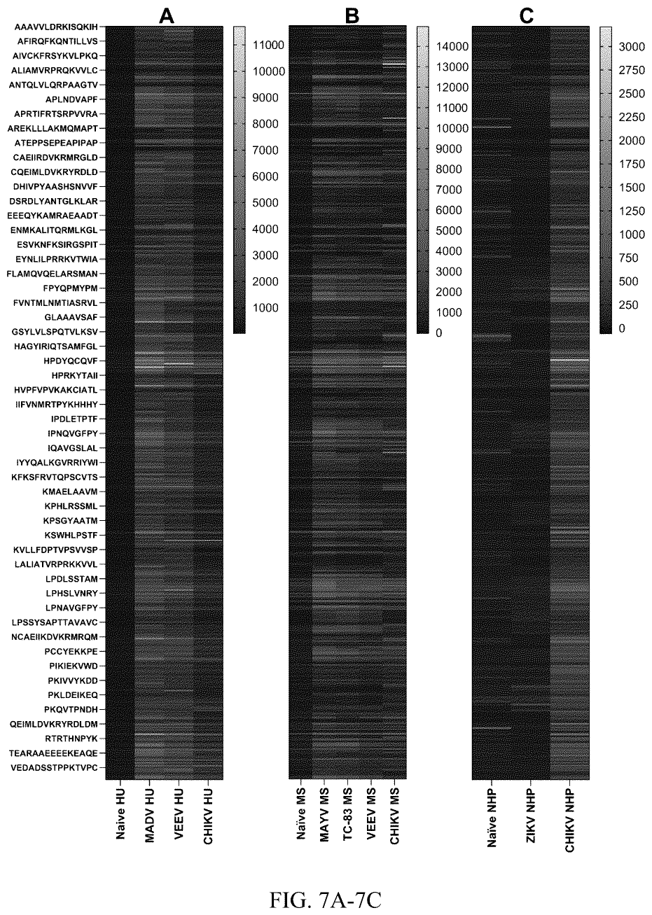

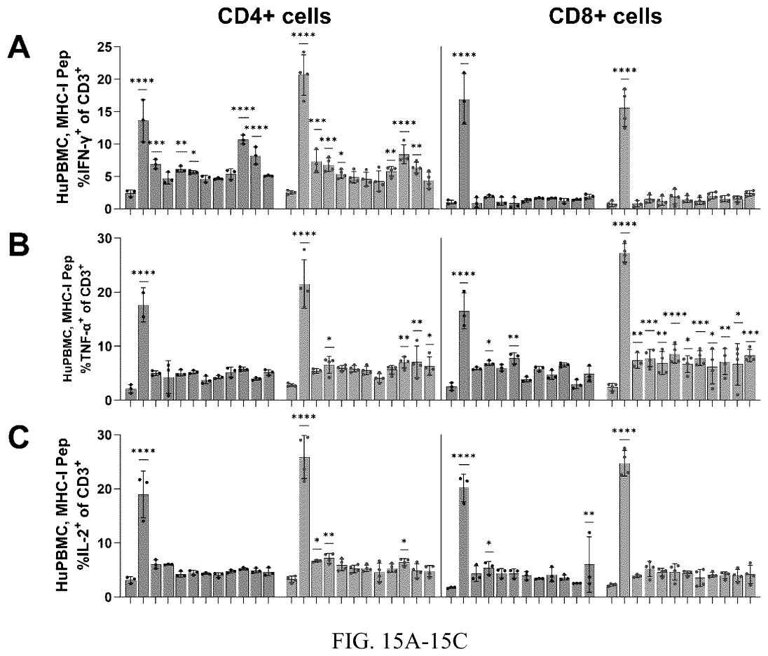

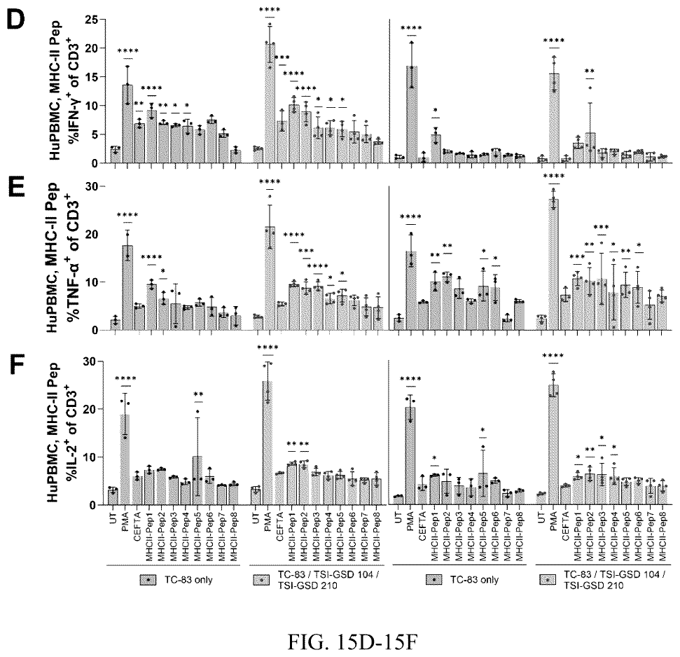

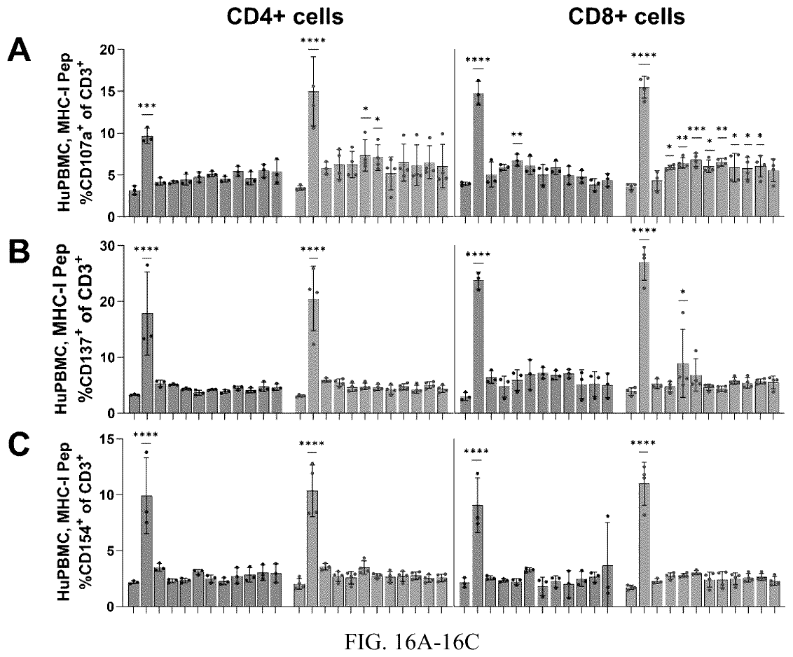

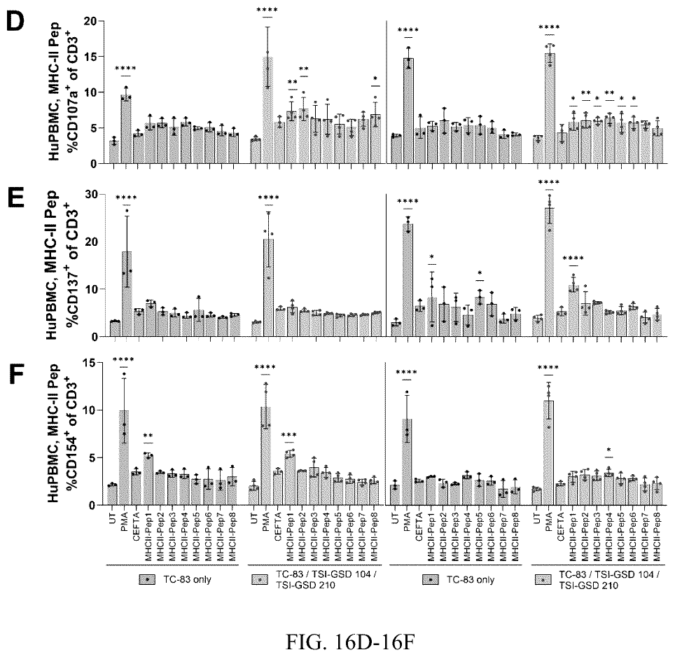

Immunoprofile of activated T-cells on human PBMCs pre-exposed to alphaviruses corresponds with in silico and in vitro analysis. In the light of the difficulty of obtaining PBMCs from patients naturally infected with different alphaviruses to corroborate the data shown here, we opt to validate our selected peptides on donors pre-exposed to other vaccines. Donors were vaccinated to one or all of the following: TC-83 (VEEV), TSI-GSD 104/inactivated PE-6 strain (EEEV), and TSI-GSD 210/inactivated CM-4884 strain (WEEV) (Table 8). Obtained PBMCs were re-stimulated in vitro as described before. Here, we observed the same pattern of cytokine secretion shown on the previous results. Similar to mouse and naïve primed T-cells (FIG. 6A), our peptides elicit a strong IFN-γ, IL-2, and TNF-α responses on PBMCs pre-exposed to alphaviruses antigens, with MHC-I Pep1, MHC-I Pep3, MHC-I Pep7, MHC-II Pep1, MHC-II Pep2, and MHC-II Pep4 eliciting the most comprehensive T-cell activation response (FIG. 6B-C). Although some donors received TC-83 only or more vaccines, the immunogenicity of the peptides did not present significant changes when analyzed by vaccine types (FIGS. 15 and 16). Interestingly, peptide MHC-II Pep1 did not present a good response against mouse PBMCs, which agrees with the pipeline classification as human top-ranked and mouse bottom-ranked (Table 4). However, this is the only peptide in a B-cell cluster that also activates T-cells in any human in vitro validation.

TABLE 8
Demographics and vaccination status of PBMC donors pre-exposed to alphaviruses proteins.

| | Donor # | 1 | 2 | 3 | 4 | 5 | 6 | 7 | 8 |
|---|---|---|---|---|---|---|---|---|---|
| Demo- | Sample ID # | mcm_0001 | mcm_0002 | mcm_0003 | mcm_0004 | mcm_0005 | mcm_0006 | mcm_0007 | mcm_0008 |
| graphics | Collection Date | Jan. 1, 2024 | Jan. 1, 2024 | Jan. 1, 2024 | Jan. 1, 2024 | Jan. 1, 2024 | Jan. 1, 2024 | Jan. 1, 2024 | Jan. 1, 2024 |
| | Ethnicity | Caucasian | Caucasian | Caucasian | African/ | Caucasian | Caucasian | Caucasian | Caucasian |
| | | | | | American | | | | |
| | Age | 43 | 39 | 35 | 40 | 38 | 38 | 66 | 41 |
| | Gender | Female | Male | Male | Male | Female | Male | Male | Male |
| Vaccine | TC-83: Venezuelan | ✓ | ✓ | ✓ | ✓ | ✓ | ✓ | ✓ | ✓ |
| status | equine encephalitis | | | | | | | | |
| | vaccine | | | | | | | | |
| | TSI-GSD | | ✓ | ✓ | ✓ | | | ✓ | ✓ |
| | 104/inactivated | | | | | | | | |
| | PE-6 strain: Eastern | | | | | | | | |
| | equine encephalitis | | | | | | | | |
| | vaccine | | | | | | | | |
| | TSI-GSD | | ✓ | ✓ | ✓ | | | ✓ | ✓ |
| | 210/inactivated | | | | | | | | |
| | CM-4884 | | | | | | | | |
| | strain: Western | | | | | | | | |
| | equine encephalitis | | | | | | | | |
| | vaccine | | | | | | | | |

| | | | | | | | | |
|---|---|---|---|---|---|---|---|---|
| TC-83 neutralization titer | 1:40 | 1:160 | 1:320 | 1:320 | 1:20 | >1:640 | >1:640 | >1:640 |

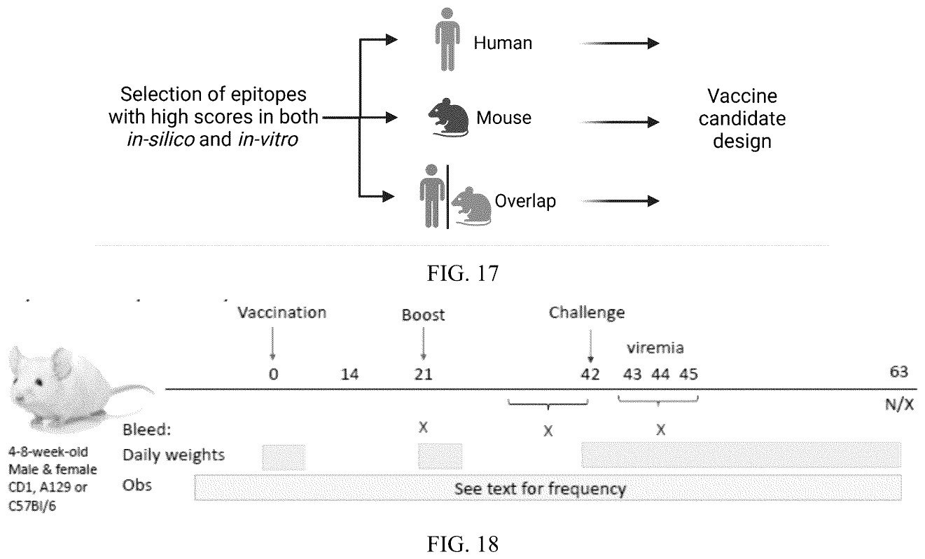

Finally, all in silico and in vitro data were included in the final run of the pipeline for the selection of candidate pan-alpha epitopes (FIG. 17). The vaccine candidates range in size from 61 to 97 amino acids. Each candidate contains 5 epitopes and 4 linkers. To overcome the limitations of in vivo experimental models, the pipeline can also be used to select species-specific immunogens, HLA-target groups, or predicted biochemical properties that impact formulation.

### B. Materials and Methods

In this study, we developed a pipeline based on viral proteomes to select T-cell immunogenic epitopes to be included in a multivalent vaccine candidate. Our pipeline has as constraints population coverage, including not only the frequency of various HLA alleles varying by geographic and ethnic background but also murine and primates HLA alleles—as those are models where the vaccine candidate will be tested. Other important prediction components are antigenicity, binding affinity, allergenicity, solubility, stability, toxicity, and physicochemical properties. All constraints are combined in a score after a series of in silico evaluations. 726 top and bottom-ranked epitopes were selected for a first reactivity evaluation using peptide microarrays, tested against pooled sera from human (n=27), mouse (n=19), and NHP (n=5) infected with different arboviruses.

Overlapping peptides were then selected and grouped by binding affinity to MHC-I or MHC-II receptors. Molecular dynamics simulations were used to confirm structure and peptide stability in the binding sites. Later, we validated the peptides in vitro by evaluating T-cell immunogenicity in different settings. First, we infected mice with MAYV or TC-83 (10 animals/group, performed in duplicates) and isolated PBMCs. These cells were then cultivated with the target peptides, and the reactivity was measured by the percentage of cells with activation surface markers and cytokine secretion. The second validation used PBMCs from healthy human donors with different ethnicities and HLA alleles (n=4/ethnic group, performed in duplicates). We differentiated and expanded antigen-specific T-cells in vitro, followed by peptide stimulation, and antigen-specific effector cytokine secretion was detected by flow cytometry. Finally, the same stimulation was performed on alphavirus pre-exposed PBMCs (derived from vaccinated donors) to confirm in vitro expansion results.

Pipeline development. We first collected 54 proteomes of sequenced alphaviruses obtained through NCBI GenBank. These samples were taken from various viral clades as shown in Table 2. This process would mimic the identification and submission of newly sequenced viral samples that can be computationally converted to proteomes and added to an expanding bio surveillance dataset. We next developed a pipeline to render vaccine candidates from these submitted proteomes. The overall schematic of the pipeline is depicted in FIG. 7 and it can be divided into the following phases:

Preliminary Epitope Identification. The pipeline initiates with a concatenation of the collected proteomes into a multi-fasta file and submission of this file to several specific epitope identification tools. For linear epitope selection, EpiDope is used to detect linear B-cell epitopes, NetMHCIPAN is used to detect linear MHC-I epitopes, and NetMHCIIPAN is used to detect linear MHC-II epitopes. For discontinuous B-cell epitopes, the pipeline first calculates folded protein structures using ESMFold2 and then submits these to DiscoTope.

All calculated epitopes are transformed into tabular output and saved to disk both for use in subsequent pipeline steps as well as for compilation in a data warehouse for ad-hoc and exploratory analyses.

Secondary Epitope Evaluation. Once initial epitope scoring has been performed, there are potentially very many candidate epitopes. For MHC-I and MHC-II epitopes, the pipeline takes the highest-ranked epitopes (as ranked by their NETMHCPAN calculated ligand elution) and performs docking simulation with MHC and TCR proteins. The threshold for what constitutes “highest-ranked” epitopes is a configurable parameter but defaults to a “Rnk EL” or “% Rank EL” value of 1.0 for NetMHCPAN and NetMHCIIPAN respectively. These epitopes are then used in TCRpMHC models to create a protein complex encompassing the peptide epitope, most predominant MHCI or MHCII peptide, and TCR alpha and beta chains. This complex is then assessed using the PDB Proteins, Interfaces, Structures, and Assemblies (ePISA) service, after which the epitope-mhc and epitope-ter interfaces are excerpted and their free-solvation energy is normalized as the number of standard deviations from the mean across all scored epitopes. This number is then used as a “score” for the stability of the TCR-peptide-MHC complex, and this is recorded in a table for inclusion in the data warehouse.

For B-Cell epitopes, the pipeline first filters scored epitopes based upon whether their originating protein is within a list of target B-cell-relevant proteins. This then results in a sub-selection of scored B-cell epitopes that are within this known set of target proteins.

Calculation of MHC Allele Frequencies. Simultaneously with the initial epitope calculation, the pipeline also computes a regional allele frequency table both for inclusion in the data warehouse and for later calculation of vaccine designs (discussed subsequently). In order to do this, the pipeline comes packaged with an extract of the Allele Frequency Net Database (allelefrequencies.net). At invocation, the pipeline accepts a parameter containing the region or regions wherein the vaccine is intended to be used. Based upon this selection and the pipeline's internal copy of the Allele Frequencies Database, each extracted subpopulation dataset is searched for regions that match the specified target regions. Then, all matching subpopulations are pooled and HLA allele frequencies are recalculated based upon this new pooled population. This recalculated table is then included in the data warehouse.

Final Vaccine Payload Evaluation. After complete execution of the pipeline, identified epitopes that have been highly scored both in-silico and in-vitro (in the top 10th percentile of average binding affinity across HLA allenes via NetMHCPanI and NetMHCPanII respectively) are used to assemble candidates. These candidates are designed via linear optimization using the JessEV epitope selection tool with the Gurobi optimizer. This tool looks to find an optimal combination of 5 epitopes (sampled from the input list without replacement) and associated linkers that preserves epitope identity with minimal or no epitope cleavage and maximal peptide representation (i.e. epitopes that map to many peptides). The JessEV program requires that all input epitopes be of the same length, so input peptides are trimmed to 9 amino acids prior to submission to JessEV. Moreover, we run JessEV for 10 iterations, removing the most immunogenic epitope at each iteration and thereby forcing the optimizer to formulate a new vaccine candidate. Note that for MHC-I epitopes, no replacement is necessary as they are already 9 amino acids in length, having been trimmed earlier in the pipeline. For MHC-II epitopes, these are 15 amino acids in length and are truncated by trimming 3 amino acids from each end (6 amino acids in total) prior to evaluation via JessEV. Retrieve the top 10 remaining candidates ranked in order of average immunogenicity across constituent T-cell epitopes.

Data Warehouse Construction. As mentioned in previous sections, each step of the pipeline creates tabular output and retains that output in order to facilitate ad-hoc or exploratory analysis. These assets include the following: (i) A table of all input proteins with their accession numbers, host organism, and phylogeny. (ii) A table of all identified B-cell epitopes (both from EpiDope and Discotope) and their predicted immunogenicity. (iii) A table linking B-cell epitopes to their associated submitted protein. (iv) A table of all identified T-cell epitopes (both from NetMHCPan and NetMHCIIPan) including their predicted immunogenicity. (v) A table linking T-cell epitopes to their associated submitted protein. (vi) A table of all TCR-pMHC multimers by MHC and peptide and their corresponding surface area and free solvation energy. (vii) A table of allele frequencies by selected region. (viii) A table of CD-Hit clusters of T-Cell epitopes including cluster assignment, centroid sequence, and centroid epitope ID. (ix) A table of CD-Hit clusters of input proteins including cluster assignment, centroid sequence, and centroid protein ID. (x) A table of submitted microarray results linking T-cell epitopes by epitope ID to measured fluorescence.

Pipeline Architecture. The pipeline described here relies on NextFlow, which is a workflow orchestration tool popular in bioinformatics. Also, the pipeline implements “containerization” which is a popular design approach that confines each analytical task to a specific, reproducible environment. Popularly, these containers can be created and used on any major operating system and can be controlled using either Docker or Singularity both of which are predominant container management tools. Importantly, NextFlow encourages a specific design pattern which this pipeline embraces. That design pattern can be succinctly summarized as follows: (i) Every task has a clearly defined input and output. (ii) Every task can be made into a container. (iii) Every task's container recipe is tracked as part of the pipeline's source code, allowing any user to rebuild and reproduce tasks and their aggregate workflows.

This results in the pipeline being represented as a directed acyclic graph (DAG) connecting each analytical operation (corresponding to a container) with intermediating channels that route the outputs of one or more source tasks into the inputs of one or more destination tasks.

Importantly, containerization and orchestration via NextFlow also means that this pipeline is highly portable in terms of where computation is performed. This means that vaccine design efforts could implement this on local servers or in any of the main cloud providers (Amazon Web Services, Microsoft Azure, Google Cloud). It is even possible to have certain analytical steps performed in separate environments as a combination of these options.

The associated GitHub repository (URL github.com/pmccaffrey6/immunoinformatics_platform) contains the source code for the pipeline itself as well as the source code required to build the task-level containers and the relevant configuration and setup code required to execute the pipeline.

In vitro validation. Mouse and nonhuman primates (NHP) sera banks were selected from laboratory collection and re-tested by PRNT for confirmation. (Table 3). Mouse sera bank is constituted of 19 samples divided by naïve, VEEV-infected, MAYV-infected, CHIKV-infected, and ZIKV-infected—as an off-target control. NHP sera bank (n=5) only includes CHIKV-infected pool sera, ZIKV-infected sera, and naïve. Animal samples were collected following UTMB policy as approved by the UTMB Institutional Animal Care and Use Committee (IACUC), protocol number 2007080 for mouse models (approved on Jul. 8, 2020), and protocol number 1912100 for NHP models (approved on Dec. 2, 2019).

We obtained 16 pre-characterized CHIKV-positive human serum from a prospective arboviral population cohort maintained in Sao Jose do Rio Preto, Brazil. The current research was conducted in compliance with Resolution 466/12 of the National Health Council of the Ministry of Health of Brazil. The study was conducted according to the guidelines of the Declaration of Helsinki and done in retrospective samples with the consent term approved by the institutional review board (IRB) of the Ethics Committee of the Faculdade de Medicina de São José do Rio Preto (protocol codes 15461513.5.0000.5415, approved on Apr. 7, 2015, and 14262619.0.0000.5415, approved on Aug. 13, 2019). Confidentiality was ensured by anonymizing all samples before data entry and analysis.

Other human samples, including Madariaga virus (MADV, n=4) and Venezuelan equine encephalitis virus (VEEV, n=6), were obtained in collaboration with the Gorgas Memorial Institute of Health Studies in Panama City, Panama. This study adopts a cross-sectional design and involves data collection conducted in October 2018 among individuals residing in the community of Aruza, Darién, Panama (protocol code 117/CBI/ICGES/23, approved on May 10, 2023). Commercially available naïve human sera, certified as reference material (Ref #NIST909C Sigma, USA), was used as control.

In vitro assessment of pan-alphavirus epitope repertoire using peptide microarray. Evaluation of the pan-alphavirus-restricted peptide repertoire was performed using PEPperCHIP© (PEPperPRINT©, Heidelberg, Germany) custom peptide microarray. All predicted peptides were included in this microarray, including T-cell overlapping epitopes and non-overlapping epitopes among different alphaviruses (control). For the microarray, 726 peptides were adsorbed in duplicate on the inside of spots located on a microarray glass slide (75.4 mm×25.0 mm×1 mm). Each slide comprehends five independent microarrays, which were later tested against different sera pools. Each pooled sera were tested in duplicates.

Staining protocol were performed according to manufacturer's instructions. The first step of the assay consisted of pre-labeling the microarray glass slide with secondary antibody to exclude background reactivity. Mouse-specific slides were coated with goat anti-mouse IgG/Cy5 (Ref #ab6563, Abcam, USA), human-specific slides were coated with goat anti-human IgG/Cy5 (Ref #ab 97172, Abcam, USA), and nonhuman primate slides were coated with goat anti-monkey IgG (Ref #617-101-012, Rockland Immunochemicals, USA) custom conjugated with Cy5 by PEPperPRINT (GER).

After the background staining, blocking and washing steps, the microarray slides were incubated for 16 hours at 2-8° C. with pre-validated sera pool from mice, nonhuman primates, and humans infected by different alphaviruses (CHIKV, MAYV, VEEV, and MADV), diluted 1:500 in staining buffer. After wash steps, microarray slide incubated with specific-secondary antibodies (described above) and anti-HA/Cy3 control antibody previously diluted (1:2,000) in staining buffer. Throughout the assay, all incubations were performed under constant agitation in an orbital agitation system (140 rpm). The PEPperCHIPC microarray slide was digitized on the Typhoon Trio (GE Healthcare, Chicago, IL, USA). Peptide microarray fluorescent signal data were quantified using MAPIX Analyzer software (Innopsys, Carbonne, FR). Results were expressed in fluorescence intensity (median florescence within replicate spots with background subtraction). Graphs were plotted using GraphPad Prism, v10.2.1.

In silico validation. The modeling of the identified MHC-I or MHC-II peptides was conducted using a customized version of AlphaFold2-Multimer (AF2M) (model version 2.3.2, available at https://github.com/google-deepmind/alphafold) and local ColabFold (model version 1.5.1, available at URL github.com/YoshitakaMo/localcolabfold/). Consistent with previous studies, we focused solely on modeling the peptide interaction MHC domain.

For the modeling with AF2M, we employed a custom sequence dataset comprising TCR and MHC sequences to speed up the MSA generation step (the customization comprises Uniref90, mgnify, seqres, small bfd and Uniprot AlphaFold datasets). AF2M was ran in multimer mode without template data cut-off, allowing structure relaxation with the Amber force field. We generated 5 models per target peptide complex, and only the top-ranked model was considered, maintaining all other parameters as default.

Modeling utilizing local ColabFold employed the alphafold2_multimer_v3 model with 20 recycles. Modeled structures were permitted to relax, while all other ColabFold parameters remained default.

To assess the quality of the generated models, we obtained the confidence scores from AF-based approaches. The multimeric confidence scores represent a combination of pTM and ipTM scores (0.8*ipTM+0.2*pTM), where higher scores indicate a more accurate modeled binding pose.

Furthermore, to evaluate whether the modeled peptides bind to the MHC in a manner consistent with known peptide: MHC complexes, thus enhancing our confidence in the model, we compared the backbone positions of modeled peptides with those of peptides bound to MHC in solved 3D structures. This comparison facilitated the identification of modeled peptides with incorrect conformation and binding modes. To conduct this evaluation, we assembled a specific dataset of structures containing MHC-I allele HLA A*02 and MHC-II molecules (in this case we did not select a specific allele since the number of available MHC-II structures is limited). The dataset only contains peptides of the same length as the identified peptides (9mers for MHC-I and 15mers for MHC-II). The MHC-I dataset was obtained from TCRModel2 (URL github.com/piercelab/tcrmodel2/tree/main/data/templates) and the MHC-II dataset was obtained from the curated PANDORA dataset (URL github.com/X-lab-3D/PANDORA). The MHC of the modeled complexes were then superposed onto each of the structures in the curated dataset, and the peptide backbone RMSD was computed. Structures with the lowest RMSD to modeled peptides are listed in Table 2. The selection of the models generated by AF2M or ColabFold for submission to MD simulations was based on the deviation from known bound complexes.

For MHC-II cases, an additional metric used to assess the modeling quality was the prediction of the peptide core binding positions using NetMHCIIpan. For this, we used the NetMHCIIpan server (version 4.0, available at URL services.healthtech.dtu.dk/services/NetMHCIIpan-4.0/) was employed, with the corresponding DRB1*07:01:01:01 allele and default options.

Molecular dynamics simulations The models of the peptides bound to the MHC obtained with the AF-based approaches served as starting points for MD simulations. Initially, each complex was protonated at pH 7.4 using the pdb2pqr30 program and propka for titration state determination. The N and C-terminal of the MHC structures was capped with NME and ACE residues with the Python PyMOL package. MD simulations were conducted using Amber 20 suite of programs with the ff14SB force field. Na+ and Cl− counter ions were added to the system using tleap to achieve net-neutralization, with an excess of salt to reach a concentration of 150 mM NaCl. Each structure was immersed into a truncated octahedral box (15 A from the solute) filled with TIP3P water. The PBRadii parameter was set to mbondi2. The system was minimized by 2500 steps of steepest descent minimization followed by 2500 steps of conjugate gradient minimization. Equilibration was performed by heating the system from 0 to 298 K over 200 ps under NVT conditions, with protein atom positions restrained. This was followed by density equilibration for 500 ps under NPT conditions without restraints. For each complex, the production run was performed at 298 K under NPT conditions for 2 ns with a time step of 2 fs, in triplicate at least. Temperature was maintained using a Langevin thermostat with a collision frequency of 5 ps-1. Hydrogen-containing bonds were constrained using SHAKE. Long-range electrostatic interactions were calculated using particle Mesh Ewald and short-range nonbonded interactions were calculated with a 9 A cutoff. The simulations were conducted using the GPU-accelerated PMEMD program, a part of AMBER 20 package.

The MD trajectories were analyzed in R using the Bio3D package. All analyses were performed after the superposition of trajectory frames by the backbone atoms of the MHC molecules using Bio3D fit.xyz function. The root-mean-square deviation (RMSD) was calculated using the Bio3D rmsd function.

Through the same MD simulation protocol, simulations of three crystallographic structures of peptide bound to MHC-I (PDB IDs: 7u21, 1duz and 5e00) were performed to be used as reference for the peptide stability. These structures were selected based on resolution criteria (<2 Å) and absence of crystallographic artifacts. To compare the positions of the peptide anchoring site P2 and PQ along the trajectories we used as reference the positions of the anchors from the structure 5hhn with the peptide bound to the MHC-I molecule.

In vivo validation with mouse model. Animals were infected using Mayaro virus (MAYV) CH strain, and the Venezuelan equine encephalitis virus (VEEV) formalin-inactivated vaccine, TC-83. TC-83 was used instead of a VEEV BSL-3 select agent strain for safety issues. However, TC-83 were previously used in VEEV pathogenic studies due to its virulence and pathogenicity similar to the original virus. Virus and vaccine strains were obtained from the World Reference Center for Emerging Viruses and Arboviruses (WRCEVA) at the University of Texas Medical Branch (Galveston, TX). The viruses were passaged once in Vero cells to generate working stocks.

Viremia and neutralization assays were performed in VERO cell line (ATCC® CCL-81™), maintained with DMEM (Gibco, USA), supplemented with 10% heat-inactivated fetal bovine serum (FBS, R&D Systems, USA) and 1% penicillin-streptomycin solution (104 U/ml and 104 μg/ml solution, respectively) (PenStrep, Gibco, USA).

Peptide synthesis. Custom peptide libraries for MHC-I and MHC-II peptides were chemically synthesized by GenScript (USA/China). Each peptide had >95% purity as determined by high performance liquid chromatography. MOG and CEFT peptide pools were commercially available at JPT Peptide Technologies (GER). Each peptide was resuspended in ddH2O or Dimethyl sulfoxide (DMSO, Sigma, USA), according to manufacturer′ recommendation. Peptides were used at a final concentration of 1 μM.

Mice infection and PBMC isolation. A cohort of 30 6-weeks old C57BL/6 mice (The Jackson Laboratory, USA) were divided by mock-infected, MAYV-infected, and TC-83-infected groups (FIG. 3A). Each group contained 5 females and 5 male animals. Animals were injected intraperitonially with 5.0 log 10 FFU and weighted daily for wellness check-up. Blood was collected retro-orbitally (RO) at the second day post-infection to assess viremia. On the 14th day post-infection, mice were euthanized for tissue harvest and blood collected by terminal cardiac puncture. Neutralization assay was used to assess specific-antibody secretion after infection.

Neutralizing antibodies were quantified by plaque reduction neutralization test (PRNT) Briefly, sera were serially diluted (1:20 up to 1:640) and incubated with 50 PFU of MAYV CH strain or TC-83 vaccine strain. After 1 h, antibody-virus solution was inoculated onto 12-well plates of Vero cells (DMEM supplemented with 2% FBS and 1% PenStrep), and non-neutralized virus was allowed to infect for one hour in a 37° C., 5% CO₂ incubator. Following this incubation, an overlay of Opti-MEM (Gibco, USA) supplemented with 2% FBS, 1% PenStrep, and 1% carboxymethylcellulose (Sigma, USA) was added to the wells and the plates were returned to the 37° C., 5% CO₂ incubator. After two days, plates were fixed with 10% buffered formalin and stained with crystal violet. Each dilution was tested in duplicity, and the number of plaque-forming units (PFU) was recorded as the average of the number observed in each test. The PRNT50 and PRNT 80 titer is the highest serum dilution able to neutralize at least 50% or 85%, respectively, of plaque formation when compared to virus-only infected control cells. All sera were incubated at 56° C. before testing to inactivate complement proteins.

Peripheral blood mononuclear cells (PBMC) were isolated by density-gradient sedimentation using Ficoll-Paque PREMIUM® (Density 1.084 g/mL, Cytiva, USA), according to manufacturer′ protocol, and cryopreserved in cell recovery media containing 10% DMSO (Gibco, USA), supplemented with 90% heat-inactivated fetal bovine serum (FBS; Hyclone Laboratories) and stored in liquid nitrogen until used in the reactivity assays.

Murine t-cell reactivity against pan-alphavirus peptides. Peptide T-cell immunogenicity was evaluated by flow cytometry. For this, cryopreserved PBMCs from infected animals were thawed with CTL Anti-Aggregate Wash™ solution (ImmunoSpot, USA), following manufacturer's recommended procedure. Cells were counted and viability measured prior incubation of 10⁶ cells/well for one hour in a 37° C., 5% CO₂ incubator in a sera-free media. After, cells were stimulated ex vivo with individual peptides for reactivity assessment. Briefly, PBMCs were cultivated with a final concentration of 1 μM of peptide, whilst the positive control was a mixture of phorbol ester, phorbol-12-myristate-13-acetate (PMA, 50 ng/ml) and ionomycin (1 μg/mL), and DMSO as negative control (UT, untreated). To all conditions were added a stimulation solution containing 2 μg/mL of anti-CD3/anti-CD28 at the beginning of the treatment. In all stimulation conditions, BD GolgiPlug™ (11 μL/mL, BD Biosciences, USA) and BD GolgiStop™ (11 μL/mL, BD Biosciences, USA) were added for the last 6-8 hours culture. Cells were incubated in a 37° C., 5% CO₂ incubator, following kinetics timepoints and harvested with 2 hs, 4 hs, 6 hs, 12 hs, and 48 hs after treatment. Each condition was tested in triplicate.

Finally, cells were harvested, washed, and immediately stained for surface markers. Staining was performed using a Zombie NIR™ Fixable Viability Kit (BD Biosciences, cat #423106), and a cocktail of antibodies directed at seven surface markers: PerCP/Cyanine 5.5 anti-mouse CD3& (clone: 145-2C11, Biolegend, USA), Brilliant Violet 510™ anti-mouse CD4 (clone: RM4-4, Biolegend, USA), Alexa Fluor® 700 anti-mouse CD8a (clone: 53-6.7, Biolegend, USA), APC anti-mouse CD137 (clone: 17B5, Biolegend, USA), Brilliant Violet 421™ anti-mouse CD25 (clone: PC61, Biolegend, USA), PE anti-mouse CD134 (OX-40) (clone: OX-86, Biolegend, USA), PE/Cyanine7 anti-mouse CD69 (clone: H1.2F3, Biolegend, USA). The cocktail of antibodies directed for cytokine staining was: Brilliant Violet 711™ anti-mouse IFN-γ (clone: XMG1.2, Biolegend, USA), PE/Dazzle™ 594 anti-mouse TNF-α (clone: MP6-XT22, Biolegend, USA), Brilliant Violet 605™ anti-mouse IL-2 (clone: JES6-5H4, Biolegend, USA). Cells were stained for specific surface molecules, fixed and permeabilized with a Cytofix/Cytoperm Kit (BD Biosciences), and then stained for specific intracellular molecules. At least 250,000 singlet events (PBMCs) were acquired, with 50,000 events on the CD3+ gate, on a FACS Symphony™ A5 (BD Biosciences, USA) and BD LSRFortessa™ Cell Analyzer (BD Biosciences, USA) analyzed using FlowJo Software, V10 (Treestar Inc., Ashland, USA). For all samples, gating was established using a combination of isotype and fluorescence-minus-one controls.

Human T-cell immunogenicity evaluation. Expansion of human alphavirus-peptide specific T-cells from healthy donors. PBMCs were thawed and expanded. Briefly, cells were plated (Day 0) with GM-CSF, IL-4, and Flt3-L overnight to mature antigen presenting cells. On Day 1, LPS, R848, and IL-1b were added with peptides (1 μM each). Starting on Day 2 and every 2-3 days after that IL-2, IL-7, and IL-15 were added. On Day 9 cells were washed, counted, and replated with anti-CD28, anti-CD49d, and desired peptides for 8 hours prior to staining for flow cytometry (FIG. 4A).

Immunogenic profile of human PBMCs pre-exposed to vaccine antigens following peptide stimulation in vitro. PBMC collection, isolation and cryopreservation were performed by the University of Texas Medical Branch (UTMB) Biorepository for Severe Emerging Infections (BSEI) team. Briefly, whole blood was collected in a Sodium Citrate treated Mononuclear Cell Preparation Tube (CPT) and PBMCs were isolated following the manufacture's protocol. Isolated PBMCs were stored in 10% DMSO in FBS at −120° C. until use. Whole blood was collected in a serum separator vacutainer and allowed to clot prior to centrifugation. Sera was aliquoted and stored at −80° C. until use.

Quantification and Statistical analysis. Several statistical methods were conducted to assess differences, correlations, and associations between groups. Due to the heterogeneous character of the data, the majority of compared groups were standardized by z-score. Flow cytometry data were collected using BD FACSDiva™ Software (BD Biosciences, USA), with raw data stored at UTMB Flow Core SharePoint cloud. Data was later processed and analyzed using FlowJo™ Software, v10.10 (BD Biosciences, USA). Graphs were plotted using GraphPad Prism software, version 10.2.1. (GraphPad Software, USA). Statistical analyses of in vitro and in vivo experiments were performed using the paired t-test, one-way ANOVA, and Kruskal-Wallis Test, as indicated in correspondent figure legend.

For analysis of in-vitro epitope testing data resulting from slide microarray against pre-exposed sera, the background fluorescence was subtracted from the per-well 635 nm fluorescence to produce a fluorescence signal for each peptide. Each epitope was also blasted against all of the input viral proteomes and marked as a representative of that organism if there was a blast result with greater than or equal to 90% identity between a query peptide and a target proteome. For analysis of T-cell and PBMC stimulation with peptides, PBMCs were tested in triplicate by ethnicity and by virus. The cytokine results were first normalized by dividing the result value by the maximum values across all tested epitopes, producing a value ranging from 0 to 1. The mean normalized cytokine level across all three replicates was first calculated. These same replicates were used to calculate the 95% Confidence Interval (CI) which was used to create upper and lower error bounds on the measurement.

### Example 2

### Initial In Vivo Test of Panalpha Vaccine

Methods Male and female CD1 mice were IM vaccinated with PanAlpha nanoparticles, gold particles, 0.9% sodium chloride (diluent) or TC-83 (experimental VEEV vaccine) on days 0 and 21. Mice were monitored for health and weights. Blood was taken for antibody and viremia quantification. Post challenge, weight, health and survival were recorded (FIG. 18).

NANOPARTZ™ Recommended Storage and Handling. Maximum shelf lifetimes for NANOPARTZ™ products by family: All bare spherical, nanorod, microgold-shelf life 6 months at 4 C; functionalized products-3 months at 4 C; organic-3 months at 4 C; and bare nanowires-6 months at room temperature. Do not freeze the nanoparticle products. In general, once conjugated, neutravidin and custom conjugations should see much greater shelf lives than when left unconjugated. For the longest shelf life, leave the functionalized products in their concentrated form and remove only what is immediately needed.

Some of products may reversibly aggregate and settle with time in storage. In these cases, these particles may be resuspended by sonication for five minutes, followed by a two minute vortex. In shipping, sometimes particles get lodged in the cap of the microcentrifuge container. A quick and easy solution is to put the tube on a vortex mixer for 3-5 seconds. Then centrifuge at less than <1000 revs/min for 30 seconds. This should recollect any particles back into the bulk reservoir. Recommended dilution buffers and methods: Adsorbed Ligand-match concentration of the absorbed ligand specified on the COA. Functionalized, in vitro-18 MEG DI water, any salt based buffer. Organic Spherical, Nsol—dilute with organic solvent of choice.

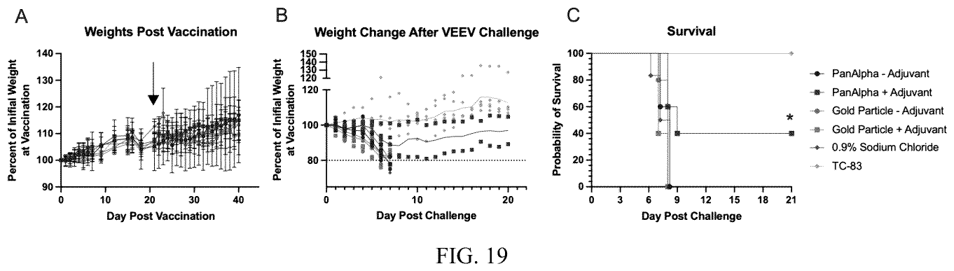

Results Mice tolerated vaccines well. No changes in weights or clinical scoring seen post vaccination. Mice were challenged with a lethal dose of Venezuelan equine encephalitis virus (VEEV), one of the viruses used to inform the PanAlpha vaccine. Changes in weights and clinical score varied with vaccine, correlated with survival (FIG. 19). PanAlpha vaccine+adjuvant vaccinated group had 40% survival. TC-83-vacinated+control had 100% survival. All other groups, including PanAlpha without adjuvant, gold particles and diluent had no survivors (FIG. 19).

1. A method for designing a vaccine candidate, comprising:
(i) conducting B-cell epitope profiling by:
(a) providing protein sequences of a plurality of proteomes of a target virus;
(b) comparing the plurality of proteomes against one or more target proteins to identify conserved protein sequences; and
(c) performing epitope detection on the conserved protein sequences to identify B-cell epitopes;
(ii) conducting T-cell epitope profiling by:
(a) predicting MHC-I and MHC-II epitopes in the plurality of proteomes;
(b) selecting a subset of epitopes based on predicted binding affinity;
(c) conducting structural analysis of the selected epitopes using three-dimensional modeling to determine free solvation energy, surface area, binding affinity, and allele frequency; and
(d) combining the free solvation energy, surface area, binding affinity, and allele frequency to generate a weighted immunogenicity score for each selected epitope, producing scored T-cell epitopes; and
(iii) designing a vaccine candidate by analyzing the scored T-cell epitopes and the B-cell epitopes to render a vaccine candidate design comprising a plurality of epitopes.

2. The method of claim 1, wherein the target virus is selected from the group consisting of alphaviruses, coronaviruses, flaviviruses, and influenza viruses.

3. (canceled)

4. The method of claim 1, wherein the B-cell epitope profiling comprises using computational tools selected from the group consisting of EpiDope, BepiPred, DiscoTope, and combinations thereof to detect linear and discontinuous B-cell epitopes.

5. The method of claim 1, wherein the T-cell epitope profiling comprises using computational tools selected from the group consisting of NetMHCpan, NetMHCIIpan, and combinations thereof to predict MHC-I and MHC-II epitopes.

6. The method of claim 1, wherein the subset of epitopes selected in step (ii) (b) comprises epitopes with a binding affinity in the top 5% of all scored epitopes.

7. The method of claim 1, wherein the structural analysis in step (ii) (c) comprises molecular dynamics simulations to assess epitope binding stability to MHC-I or MHC-II molecules.

8. The method of claim 7, wherein the molecular dynamics simulations are performed using a computational tool selected from the group consisting of AlphaFold2-Multimer, ColabFold, Amber, and combinations of one or more thereof.

9. The method of claim 1, wherein the allele frequency is calculated based on a regional allele frequency table derived from a database of HLA allele frequencies for a target population.

10. The method of claim 9, wherein the target population is selected based on geographic or ethnic background, including populations in South America, North America, Africa, Asia, or Europe.

11. The method of claim 1, further comprising validating the vaccine candidate design in vitro by evaluating epitope reactivity using peptide microarrays tested against sera from human, mouse, or nonhuman primate subjects pre-exposed to the target virus.

12. The method of claim 11, wherein the in vitro validation comprises assessing T-cell immunogenicity by measuring activation markers and cytokine secretion in peripheral blood mononuclear cells (PBMCs) from subjects pre-exposed to the target virus or vaccinated with a related antigen.

13. The method of claim 12, wherein the activation markers are selected from the group consisting of CD69, CD25, OX-40, CD107a, CD137, CD154, and combinations thereof, and the cytokines are selected from the group consisting of IFN-γ, TNF-α, IL-2, and combinations thereof.

14. The method of claim 1, further comprising validating the vaccine candidate design in vivo by administering the vaccine candidate to a mouse model and assessing immune response and protection against challenge with the target virus.

15. The method of claim 1, wherein the vaccine candidate design is optimized for a specific species, HLA allele group, or biochemical property selected from the group consisting of antigenicity, solubility, stability, toxicity, and allergenicity.

16. The method of claim 1, wherein the method is implemented using a computational pipeline orchestrated by Nextflow with containerization of analytical tasks.

17. The method of claim 16, wherein the computational pipeline is executed on a high-performance computing (HPC) cluster or a cloud computing environment selected from the group consisting of Amazon Web Services, Microsoft Azure, Google Cloud, and combinations thereof.

18. The method of claim 16, wherein the computational pipeline has a runtime of approximately 48 hours using 256 computer cores, 1000 GB of RAM, and at least one Nvidia Tesla A100 80 GB GPU.

19. A vaccine candidate composition comprising a plurality of epitopes identified using the method of claim 1.

20. The vaccine candidate composition of claim 19, wherein the plurality of epitopes comprises at least one MHC-I epitope and at least one MHC-II epitope.

21. (canceled)

22. The vaccine candidate composition of claim 19, wherein the plurality of epitopes comprises at least one epitope selected from the group consisting of APLQHTAPF (SEQ ID NO:1), APRRRVGGF (SEQ ID NO:2), FPSISTTAW (SEQ ID NO:3), HPQHHAQTF (SEQ ID NO:4), HPQLHAQTF (SEQ ID NO:5), KPDYRCQTY (SEQ ID NO: 6), PCCYEKGPE (SEQ ID NO:7), PCCYEKQPE (SEQ ID NO:8), PDDQDTGSE (SEQ ID NO:9), APCSLVSYHGYYILA (SEQ ID NO:10), CYMFATARRKCLTPY (SEQ ID NO:11), HAGYIRIQTSAMFGL (SEQ ID NO:12), IIFVNMRTPYKHHHY (SEQ ID NO:13), KALITQRMLKGLGHY (SEQ ID NO:14), KLFLAKSATRSIVER (SEQ ID NO:15), LARRFSSFRAVTVRC (SEQ ID NO:16), LASCYMFATARRKCL (SEQ ID NO:17), and combinations thereof.

23.-47. (canceled)
